(19) United States
(12) Patent Application Publication
(10) Pub. No.: US 2024/0123113 A1
(43) Pub. Date: Apr. 18, 2024

(54) Pectin Compositions and Methods of Use

(71) Applicant: The Brigham and Women`s Hospital, Inc., Boston, MA (US)

(72) Inventor: Steven James Mentzer, Boston, MA (US)

(21) Appl. No.: 18/276,540

(22) PCT Filed: Feb. 9, 2022
(86) PCT No.: PCT/US2022/015820

§ 371 Date: Aug. 9, 2023

Related U.S. Application Data
(60) Provisional application No. 63/147,601, filed on Feb. 9, 2021.

(51) Int. Cl. A61L 24/08; A61L 24/00; C09J 105/06

(52) U.S. Cl. CPC A61L 24/08; A61L 24/0015; A61L 24/0042; C09J 105/06; A61L 2430/16

(57) ABSTRACT

The present disclosure describes bioadhesive polymer composition comprising: a first film comprising a polymer comprising: i) high-methoxyl pectin (HMP) and ii) an initial water content ranging from about 37% to about 43% (w/w); and a second film comprising a polymer comprising: i) HMP and ii) an initial water content ranging from about 9% to about 13% (w/w), wherein the first and second films are adhered to each other. Methods of preparing the bioadhesive polymer compositions and methods of sealing an ocular injury in an eye of a subject are also described.

### CLAIM OF PRIORITY

This application claims the benefit of U.S. Provisional Patent Application Ser. No. 63/147,601 filed on Feb. 9, 2021, the entire contents of which are hereby incorporated by reference.

### FEDERALLY SPONSORED RESEARCH

This invention was made with government support under grant numbers HL134229 and HL007734 awarded by the National Institutes of Health and under grant number DE-SC0015662 awarded by the United States Department of Energy. The government has certain rights in the invention.

### TECHNICAL FIELD

The present disclosure describes bioadhesive polymer compositions including one or more films comprising a polymer comprising high methoxyl pectin (HMP) and water. The disclosure also describes methods of treating an ocular injury in a subject in need thereof with these compositions and methods of preparing the compositions.

### BACKGROUND

Despite remarkable improvements in cataract surgery, postoperative endophthalmitis remains a concern. Detailed studies indicate that the incidence of ocular infection after cataract surgery is 0.08 to 0.014%. The reason for this low but persistent incidence of endophthalmitis is that cataract surgery is difficult to perform under sterile conditions. The cornea is always exposed to the outside world—bacterial flora are found in the conjunctival sac, meibomian glands, eye lashes, palpebral skin as well as other parts of the eye. In this non-sterile milieu, surgical compromise of the corneal barrier can lead to infectious complications.

Sutureless clear corneal incisions (CCIs) are commonly performed for cataract surgery. CCI has many advantages; for example, CCI eliminates suture-related corneal astigmatism, shortens operative time, reduces the cost of surgery, and simplifies postoperative management. However, CCIs are not typically associated with definitive corneal wound closure. Forgoing definitive closure is associated with a small risk of wound leak, bacterial translocation and ocular infection.

In patients with a wound leak, the corneal barrier can be further compromised by unpredictable variation in postoperative intraocular pressure (TOP). Increases in IOP may occur in postoperative patients. Patients may inadvertently squeeze their eyelid or rub their eyes; these activities can increase IOP to as high as 110 mmHg. Decreases in IOP are a particular risk for wound infection. IOP less than 5 mmHg are susceptive to wound separation; even IOP less than 10 mmHg have been associated with wound separation. Moreover, hypotony is unpredictable at the time of surgery. Low IOP can be present during the first 24 hours after surgery even in the presence of a seemingly watertight sutureless incision.

These observations suggest the desirability of a corneal wound sealant capable of preventing transcorneal fluid movement without compromising the advantages of CCI; namely, a wound sealant that is simple, efficient, and effective.

### SUMMARY

Certain aspects of the present disclosure are directed to bioadhesive, pectin-based polymer composition and method of manufacture and use comprising any feature described herein, either individually or in combination with any feature, in any configuration.

Certain aspects of the present disclosure are directed to bioadhesive polymer compositions comprising a first film comprising a polymer comprising: i) high-methoxyl pectin (HMP) and ii) an initial water content ranging from about 37% to about 43% (w/w); and a second film comprising a polymer comprising: i) HMP and ii) an initial water content ranging from about 9% to about 13% (w/w), wherein the first and second films are adhered to each other, and wherein the first film is in a gel phase and the second film is in a glass phase.

In some embodiments, the HMP is present at a concentration of about 50% (w/w) to about 100% (w/w) in each of the first and second films. In some embodiments, an adhesivity of the first and second films to each other ranges from about 22 newton (N) to about 28 N. In some embodiments, the first film and the second film have a cross-grain texture at an interface between the first and second films. In some embodiments, the initial water content of the first and second films is a water content before and during an initial period of time after the first and second films are adhered to each other, wherein the initial period of time is about 10 minutes at most.

In some embodiments, a final water content of the first and second films is a water content after the initial period of time. In some embodiments, the final water content ranges from about 20% to about 30% (w/w). In some embodiments, the first and second films exhibit interdiffusion of water. In some embodiments, a diffusion coefficient of water through the first and second films is about 2.5-fold to about 5-fold slower than a self-diffusion coefficient of water. In some embodiments, the first and second films exhibit interdiffusion of HMP. In some embodiments, a water content at an interface between the first and second films differs from the water content elsewhere in a bulk of the first and second films. In some embodiments, the first and second films have a work of cohesion ranging from about 100 J/m2 to about 150 J/m2. In some embodiments, the first film has thickness of about 62 μm, and the second film has thickness of about 40 μm.

Certain aspects of the present disclosure are directed to bioadhesive polymer compositions comprising a first film comprising a polymer comprising: i) about 50% (w/w) to about 100% (w/w) high-methoxyl pectin (HMP) and ii) water; and a second film comprising a polymer comprising: i) about 50% (w/w) to about 100% (w/w) HMP and ii) water, wherein the first and second films are adhered to each other.

In some embodiments, i) the first film is in a glass phase, and the second film is in a gel phase; ii) the first film is in a glass phase, and the second film is in a glass phase; or iii) the first film is in a gel phase, and the second film is in a gel phase.

Certain aspects of the present disclosure are directed to methods of preparing a conjoined bioadhesive polymer composition, the method comprising: providing a first film comprising a polymer comprising: i) high-methoxyl pectin (HMP) and ii) an initial water content ranging from about 37% to about 43% (w/w); providing a second film comprising a polymer comprising: i) HMP and ii) an initial water content ranging from about 9% to about 13% (w/w); and compressing the first film and the second film, thereby producing the conjoined bioadhesive polymer composition, wherein the first film is in a gel phase and the second film is in a glass phase.

In some embodiments, the HMP is present at a concentration of about 50% (w/w) to about 100% (w/w) in each of the first and second films. In some embodiments, each of the first and second films has a water content ranging from about 20% to about 30% (w/w) after the compressing step. In some embodiments, the first and second films are compressed with a force of about 10 N to about 80 N. In some embodiments, the first and second films are compressed for about 10 seconds to about 12 hours.

Certain aspects of the present disclosure are directed to methods of sealing an ocular injury in an eye of a subject, the method comprising: providing a bioadhesive film comprising a polymer comprising: i) high-methoxyl pectin (HMP) and ii) water; and contacting the eye of the subject with the bioadhesive film; and applying pressure to the bioadhesive film, thereby sealing the ocular injury in the eye of the subject.

In some embodiments, the IMP is present at a concentration of about 50% (w/w) to about 100% (w/w). In some embodiments, the water is present at a concentration of about 10% (w/w) to about 30% (w/w). In some embodiments, the bioadhesive film reaches 80% of maximal adhesion to the eye within at least about 5 seconds of contact. In some embodiments, the ocular injury is a corneal incision or an injury caused by an ocular surgery. In some embodiments, the bioadhesive film is flexible, translucent, bioabsorbable, fracture-resistant, or any combination thereof. In some embodiments, the bioadhesive film exhibits minimal optical distortion. In some embodiments, the bioadhesive film adheres to the eye of the subject with an adhesion strength of at least about 3 N. In some embodiments, the bioadhesive film has a thickness of about 40 μm to about 200 μm. In some embodiments, after the bioadhesive film seals the ocular injury, the eye is resistant to a pressure fluctuation ranging from about 50 mmHg to about 280 mmHg. In some embodiments, the bioadhesive film further comprises a therapeutic agent, and wherein the therapeutic agent is an anti-bacterial agent, an anti-inflammatory agent, a growth factor, or any combination thereof.

The terms “subject” or “patient” as used herein refer to any mammal (e.g., a human or a veterinary subject, e.g., a dog, cat, horse, cow, goat, sheep, mouse, rat, or rabbit) to which a composition or method of the present disclosure may be administered, e.g., for experimental, diagnostic, prophylactic, and/or therapeutic purposes. The subject may seek or need treatment, require treatment, is receiving treatment, will receive treatment, or is under care by a trained professional for a particular disease or condition.

As used in the specification and the appended claims, the singular forms “a,” “an” and “the” include plural referents unless the context clearly dictates otherwise.

The term “ocular injury,” as used herein, can include ulcers, lacerations, defects, perforations, or intentionally performed incisions (e.g., as is done in surgery) of the cornea or sclera.

As used herein, the term “therapeutic agent” is any molecule or atom which is encapsulated, conjugated, fused, dispersed, embedded, mixed, or otherwise affixed to any of the compositions described herein and is useful for a disease therapy.

As used herein, the expression “pharmaceutically acceptable” applies to a composition which contains composition ingredients that are compatible with other ingredients of the composition as well as physiologically acceptable to the recipient (e.g., a mammal such as a human) without the resulting production of excessive undesirable and unacceptable physiological effects or a deleterious impact on the mammal being administered the pharmaceutical composition. In some embodiments, a composition for use comprises one or more carriers, useful excipients, and/or diluents.

As used herein, the term “biodegradable” refers to a substance which may be broken down by microorganisms, or which spontaneously breaks down over a relatively short time (within about 14 days to about 6 months) when exposed to environmental conditions commonly found in nature. For example, the compositions described herein may be degraded by enzymes which are present in the body (e.g., the ocular environment).

As used herein, the term “adhesion” means an abnormal attachment between two tissues or between a tissue and an organ that form after an inflammatory stimulus such as surgical or other trauma.

As used herein, the terms “adhesion inhibition” and “anti-adhesion” refer to reducing the formation of post-surgical adhesions, e.g., in the form of a scar and/or a fibrous band, between traumatized tissues, and between traumatized and non-traumatized tissues.

As used herein, the term “bioadhesive” refers to a composition that can securely bind to living tissue.

As used herein, the term “bioresorbable” when referring to a composition means that the composition can be reabsorbed and eliminated from the body.

As used herein, the term “biocompatible” means a composition that is physiologically acceptable to a living tissue and organism.

As used herein, a “pectin” is any one of a family of galacturonic acid-rich polysaccharides including homogalacturonan, rhamnogalacturonan I, and the substituted galacturonans rhamnogalacturonan II (RG-II) and xylogalacturonan (XGA), as described in Mohnen, “Pectin Structure and Biosynthesis,” Current Opinions in Plant Biology, 11:266-277, 2008. High methoxyl pectins and amidated pectins are variations of the pectin family.

As used herein, the terms “high-methoxyl pectin,” “high-methyl pectin,” and high methyl ester-pectin” are used interchangeably.

Ranges may be expressed herein as from “about” one particular value, and/or to “about” another particular value. When such a range is expressed, another embodiment includes from the one particular value and/or to the other particular value. The use of the term “about,” as used herein, refers to an amount that is near the stated amount by about 10%, 5%, or 1%, including increments therein. For example, “about” can mean a range including the particular value and ranging from 10% below that particular value and spanning to 10% above that particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Where values are described in the present disclosure in terms of ranges, endpoints are included. Furthermore, it should be understood that the description includes the disclosure of all possible sub-ranges within such ranges, as well as specific numerical values that fall within such ranges irrespective of whether a specific numerical value or specific sub-range is expressly stated.

Other features and advantages of the present disclosure will be apparent from the following detailed description and figures, and from the claims.

Various embodiments of the features of this disclosure are described herein. However, it should be understood that such embodiments are provided merely by way of example, and numerous variations, changes, and substitutions can occur according to those skilled in the art without departing from the scope of this disclosure. It should also be understood that various alternatives to the specific embodiments described herein are also within the scope of this disclosure.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features and advantages of the invention will be apparent from the following detailed description and figures, and from the claims.

### DESCRIPTION OF DRAWINGS

FIGS. 1A-1F show the polymer-polymer adhesion testing between glass- and gel-phase films. FIG. 1A is a graph showing curing time as a function of water content. Liquid pectin solutions (3% w/w) were cured in a low-humidity environment; the progressive evaporation of water created the gel phase and glass phase films used in these studies. FIG. 1B is a photographic image showing a film in the gel phase (e.g., 38-41% w/w W_{c}); films in gel phase were soft and flexible. FIG. 1C is a photographic image showing a film in the glass phase (e.g., 10-13% w/w W_{c}); films in the glass phase were hard and brittle. FIG. 1D is a photographic image showing an exemplary experimental setup. Polymer-polymer adhesion experiments were performed with a custom fixture composed of a flat-ended cylindrical probe and parallel fixture surface for a preset development time. The cylindrical probe compressed the two polymers followed by the separation of the probe from the surface by an applied tensile load. FIG. 1E is a schematic illustrating an exemplary experimental setup. The fixture ensured that the radius of contact was defined as the radius of the probe (a), and the thickness of the contacted polymers was h. The pre- and post-experimental geometries were nearly equivalent for large values of a/h with minimal edge effects. FIG. 1F is a graph showing data from a standard adhesion test. The data reflected the compression force applied by the probe (i), the development time (ii) and the strength of the polymer-polymer adhesion during probe separation (iii).

FIGS. 2A-2C show the adhesive strength of glass and gel phase films. FIG. 2A is a set of graphs showing adhesion data. Glass films (10-13% w/w W_{c}) demonstrated virtually no adhesion to other glass films. Gel phase films (38-41% w/w W_{c}) demonstrated greater adhesion when paired with either a glass phase film or another gel phase film. Representative data is shown; arrows demonstrate peak adhesion. FIG. 2B is a graph showing adhesion strength data. The adhesion strength of glass-glass films was significantly less than gel-glass or gel-gel interactions (p<0.0001) (N=40 films; error bars 1 SD). Both orientations of the gel-glass and glass-gel films in the adhesion test fixture were evaluated to control for fixture or gravity influences. FIG. 2C is a graph showing the relative adhesion strength of different mixtures. The area of the bubbles reflects relative adhesion strength of the different mixtures scaled to 100.

FIGS. 3A-3B show the water diffusion between pectin films. The diffusion of water was assessed between adherent gel-phase films (40% w/w W_{c}) and glass-phase films (10% w/w W_{c}). FIG. 3A is graph showing percentage of water content as a function of time. Water content was assessed at time=0, t=5 min and t=10 min. Each column represents the mean water content of 4 films±1 SD. FIG. 3B is graph showing water content assessed at various intervals from t=0 to t=1 h. Each data point represents the mean water content of four films±1 SD. Since the diffusion coefficient of water through the pectin medium at 25° C. is unknown, the data is plotted relative to theoretical diffusion coefficients of 0.00092 mm²/s (dashed line), 0.00183 mm²/s (solid line) and 0.00732 mm²/s (dotted line) for comparison.

FIGS. 4A-4D show the effect of polymer-polymer compression and development time on adhesion strength. Paired gel phase films were compressed for 60 s at compression forces ranging from 10 N to 80 N. FIG. 4A is a graph showing representative data for gel phase films compressed at 30 N. The integral of the compressive force over time (impulse) is shaded gray for presentation purposes. FIG. 4B is a graph showing adhesive strength as a function of compression force. The adhesive strength increased with increasing compression force. The data reflect the adhesion strength in mean N±1 SD (N=70 films). A linear curve fit (R2=0.956) is shown (dotted line). To evaluate the effect of time on polymer-polymer adhesion strength, paired gel phase polymers were compressed at 20 N for time intervals ranging from 10 s to 12 h. The films were maintained in a controlled humidity microenvironment to maintain polymer hydration. FIG. 4C is a graph showing representative data demonstrating 20 N compression for 3600 s (1 h) followed by probe withdrawal (arrow). FIG. 4D is a graph showing most of the adhesive strength increase was observed within 10 min of contact. The data reflect the adhesion strength in N±1 std. dev. (N=45 films). A logarithmic curve fit (R²=0.913) is shown (dotted line).

FIGS. 5A-5B show the fracture mechanics of pectin polymer-polymer films. The fracture mechanics of two adherent gel phase films (conjoined) was compared to two stacked films (two-layer) and a cured film with double pectin content (double). The films in all three conditions had virtually-identical pectin content (mean 88±1 mg) and water content (mean 13±0.4%) when fractured. FIG. 5A is a graph showing the fracture mechanics of different pectin films. The pectin films were loaded (1 mm/s) with a 5-mm spherical stainless-steel probe until fracture. The peak force was recorded as burst strength. The two-layer films typically demonstrated a bimodal pattern consistent with independent fracture of the two stacked films. Burst strength, reflecting the peak force required for fracture, was not significantly different between the three films (p>0.01). FIG. 5B is a graph showing the work of cohesion of the different pectin films. The decreased stiffness and increased extensibility of the conjoined films was associated with a significantly increased work of cohesion (J/m²) (p<0.01).

FIGS. 6A-6D are scanning electron microscopy (SEM) images of pectin polymer-polymer films. Conjoined films were produced by compressing two gel phase films at 20 N for 60 s followed by curing to the glass phase. The glass phase conjoined films were sharply divided at the midpoint and examined by SEM. FIG. 6A is an SEM image showing a polymer-polymer interface is at the cut surface. FIGS. 6B, 6C, and 6D are high resolution SEM images showing various views of the conjoined films. Evidence for a cross-grain effect is illustrated by the arrows.

FIGS. 7A-7D shows physical and optical properties of pectin tape. FIG. 7A is a photographic image of pectin tape demonstrating how pectin tape is flexible and semi-transparent. FIG. 7B is a photographic image of pectin tape showing pectin tape causes some discoloration, but no distortion of an Amsler grid. FIG. 7C is a graph showing the absorbance of light of a pectin film. The slight yellow color of the pectin was not associated with a significant impact on light absorbance in the visual range (black line, circle). Blue, red, yellow and orange transparent optical standards are shown for comparison. FIG. 7D is a photographic image of pectin tape sealing a corneal incision in the bovine cornea (white arrow).

FIGS. 8A-8D show the adhesive strength of pectin tape. FIG. 8A is a schematic illustrating an exemplary custom experimental design. Pectin tape was mounted on a cylindrical probe (yellow). The bovine cornea (white) was fixed to a foam hemispherical mounting fixture. The pectin tape and cornea were compressed at 5 N for 60 s followed by probe withdrawal at 0.5 mm/s. The force required for probe withdrawal was measured at 500 pps. FIG. 8B is a graph showing adhesion data. The adhesion curves demonstrated a peak force of greater than 3 N and evidence of a prominent debonding curve (arrow). FIG. 8C is a graph showing the peak force of the pectin tape was significantly greater than 3 other biopolymers including NCF, hyaluronate and CMC (p<0.05). FIG. 8D is a graph showing the work of adhesion of pectin films compared to other biopolymers. The comparison of the work of adhesion of pectin, reflecting the area under the adhesion curve, was also significant (p<0.05). The boxes span the interquartile range with the median marked with an X and the whiskers defining the data range.

FIGS. 9A-9D show the peel strength of pectin tape. FIG. 9A is a schematic demonstrating an exemplary experimental design. The bovine cornea (white) was fixed to a foam hemispherical mounting fixture. The pectin tape and cornea were compressed at 5N for 60 s followed by probe withdrawal at 0.5 mm/s. The force required for probe withdrawal was measured at 500 pps; simultaneous video recordings (12 megapixels) were obtained at 30 fps with time-based correction. Peel angles were measured after processing with standard MetaMorph filters (Molecular Devices). FIG. 9B is a photographic image showing exemplary peel angles indicated by arrows. FIG. 9C is a graph showing how adhesion curves demonstrated a peak force greater in the low angle region of the adhesion curves. FIG. 9D is a graph showing how the comparison of peel adhesion strength was significantly greater at 30 degrees than 60 or 120 degrees (p<0.05). The boxes span the interquartile range with the median marked with an X and the whiskers defining the data range.

FIGS. 10A-10E show the pressure resistance of pectin tape. FIG. 10A is a schematic demonstrating an exemplary experimental design. Separate custom fixtures were used for testing elevated and reduced pressures across the bovine cornea. In both assays, a progressive transcorneal pressure gradient was created; the yield point was identified by a statistically significant drop in the pressure gradient. FIG. 10B is a graph showing how in the increased pressure condition, the yield point was identified by a significant loss in transcorneal pressure rise (arrow). FIG. 10C is a graph showing how, similarly, the decreased pressure condition, the yield point was identified by a decrease in the negative transcorneal pressure gradient (arrow). Sudden or complete sealant failure was not observed. The peak pressure gradient in the increased (FIG. 10D) and decreased (FIG. 10E) pressure conditions were highly significant relative to the no tape control (p<0.001)(CCI=clear corneal incision; +PT=plus pectin tape). The boxes spans the interquartile range with the median marked with an X and the whiskers defining the data range.

FIGS. 11A-11D show scanning electron microscopy (SEM) images of the bovine cornea and pectin tape. The pectin tape (arrows) is adherent to the bovine cornea (c). FIG. 11A shows an overview of the bovine cornea (c) with adherent pectin tape. FIGS. 11B, 11C, and 11D show the pectin-corneal interface (arrows).

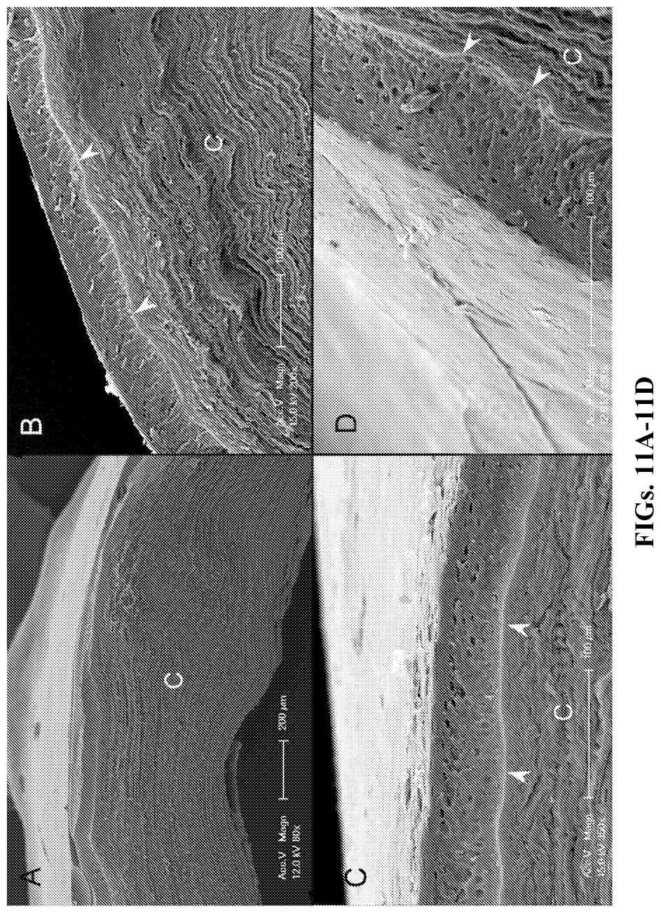

### DETAILED DESCRIPTION

The compositions described herein include bioadhesive polymer (e.g., pectin-based) compositions. In some examples, the compositions described herein are used for sealing a wound (e.g., an ocular injury). Methods of preparing these compositions are also provided herein. Some embodiments of the compositions and methods described herein may provide one or more of the following advantages.

Certain embodiments of the present disclosure include bioadhesive polymer compositions, some of which are referred to herein as pectin tape. As discussed above, there is currently a need for a corneal wound sealant that is simple, efficient, and effective. The compositions and methods of the present disclosure address this need. For example, in some embodiments, the compositions and methods described herein have adhesive strength and pressure resistance when adhered to a tissue (e.g., a visceral tissue and/or an ocular tissue). In some embodiments, the compositions of the disclosure help prevent wound leakage after corneal surgery. The bioadhesive polymer compositions, e.g., pectin tape, can be capable of limiting wound leakage despite wide ranges in intraocular pressure (IOP).

Some embodiments described herein may provide a flexible bioadhesive polymer composition. For example, in addition to limiting wound leaks, the convenience of the bioadhesive polymer compositions described herein can also increase surgical flexibility. With an effective sealant, such as those described herein, corneal surgery would not be limited to complex incisional architecture and may permit larger or secondary incisions when necessary without the increased risk of perioperative morbidity.

Some embodiments described herein may provide methods of sealing a wound that are simple and time efficient. For example, the methods of the disclosure can require an application routine that is reminiscent of the application of pressure sensitive adhesives (e.g., Scotch® tape). In other words, adhesion of the bioadhesive polymer compositions described herein can develop in seconds. Thus, in some embodiments, the compositions and methods of the disclosure provide a user (e.g., a clinician) with an efficient method to easily seal a wound in a tissue that requires no additional steps other than contacting the bioadhesive film(s) with the tissue of interest. For example, in some embodiments, the methods described do not require a user to mix various components. In some embodiments, the bioadhesive polymer compositions of the disclosure do not have a limited time window within which a user must apply the composition to a tissue before an undesirable change to the composition occurs (e.g., unwanted solidification, unwanted separation of components, or the like).

Moreover, some embodiments described herein may provide bioadhesive polymer compositions with optimum optical properties (e.g., optical transparency). In some embodiments, the compositions and methods of the disclosure may further provide patients with an ocular sealant that is flexible, translucent, fracture-resistant, and offers minimal optical distortion. Furthermore, the bioadhesive polymer compositions of the disclosure have a thickness that is likely to be less of a wound irritant than the typical application of cyanoacrylate or polyethylene glycol-based adhesives. Thus, in some embodiments, the compositions and methods of the disclosure may be well suited to be applied to the eye for extended periods of time.

In addition, some embodiments described herein may provide a bioabsorbable sealant. For example, in contrast to sutures, the bioadhesive polymer compositions described herein are bioabsorbable so there is no need for postoperative removal of any compositions. Furthermore, when used as an optical sealant, there is a reduced or non-existent risk of sealant-related astigmatism.

Furthermore, the bioadhesive polymer compositions described herein are biocompatible and safe. For example, the bioadhesive polymer compositions include HMP, which has been widely recognized as a harmless food additive in North America and Europe. In the United States, pectin is affirmed GRAS (Generally Recognized as Safe) as defined in the Code of Federal Regulations (21CFR184.1588).

Additionally, some embodiments described herein may provide bioadhesive polymer compositions that may have tunable properties. In some embodiments, physical properties (e.g., extensibility, stiffness, work of cohesion, adhesivity, shape, thickness, or any combination thereof) of the bioadhesive polymer compositions described herein can be easily optimized. For example, extensibility can be increased, stiffness can be decreased, and work of cohesion can be increased relative to native polymers by adhering one or more films to a first film, thereby resulting in conjoined films. In another example, the adhesivity of the pectin-based films can be controlled by varying the concentration of pectin in the composition. For example, lowering a concentration of pectin can decrease surface adhesivity of the pectin-based films. Furthermore, the shape and thickness of the pectin-based films can be controlled by controlling the shape and/or dimensions of the mold in which the pectin-based polymer solution is cured. Additionally, the phase of the pectin-based films can be controlled by varying the water content in the bioadhesive polymer composition. For example, lowering the water content in the bioadhesive polymer composition can induce gel and glass phase transitions. Thus, in some embodiments, the compositions and methods of the disclosure may provide a flexible sealant that can be optimized for various tissue types and injuries.

### Compositions

The present disclosure features bioadhesive polymer compositions that can include one or more films comprising high-methoxyl pectin (HMP) and water. In some embodiments, the bioadhesive polymer compositions can include two films that are adhered to each other, each film comprising high-methoxyl pectin (HMP) and water.

Pectins are a family of plant cell wall polysaccharides and/or glycan domains that consist mainly of esterified D-galacturonic acid residues in (1→4) chains. Pectins differ from typical pressure sensitive adhesives, as they do not bind to most non-biologic compounds. However, they selectively and strongly bind to the mesothelial glycocalyx, which is likely the result of a mechanism of interdiffusion or interpenetration, e.g., the entanglement of branched chain polysaccharides based on chemical bonds and weak chemical interactions.

Pectins can vary in molecular weight, cross-linking density (determined by multi-angle laser light scattering), and chemical groups (e.g., hydroxyl, amine, sulfur and carboxyl groups). The polysaccharides that make up pectin are generally grouped into three major types: homogalacturonan (HG), rhamnogalacturonan I (RG-I), and the substituted galacturonanrhamnogalacturonan II (RG-II). Some plant cell walls also contain additional substituted galacturonans, known as apiogalacturonan (AGA) and xylogalacturonan (XGA). Thus, pectins are often defined by their source, e.g., citrus pectin (which was used in the examples described herein). The most common categories of pectin vary with respect to amidation and methoxylation; however, all pectins appear to be biocompatible. When exposed to calcium, pectin forms egg box-like structures that facilitate the immobilization of substances within the gel structure.

The pectins used to prepare the polymer compositions described herein are preferably high-methoxyl pectins (HMP), which can be obtained commercially (e.g., from Cargill, Minneapolis, Minn., USA). The proportion of galacturonic acid residues in the methyl ester form determines the degree of methoxylation. HMPs are defined herein as those pectins with a degree of methoxylation greater than 50%; low-methoxyl pectins (LMP) are defined herein as those pectins with a degree of methoxylation of less than 50%. The LMP were also tested as non-amidated (LMC) and amidated (LMA) variants. While there are several different methods of determining the degree of methoxylation of pectin, the quantitative test used in the Examples to determine the degree of methoxylation of a given pectin is an NMR-based method.

Disclosed herein, in certain embodiments, are bioadhesive polymer compositions comprising a film comprising a polymer comprising HMP and water. In some embodiments, HMP is present in the composition at a concentration ranging from of about 50% (w/w) to about 100% (w/w) (e.g., about 50% (w/w) to about 60% (w/w), about 50% (w/w) to about 70% (w/w), about 50% (w/w) to about 80% (w/w), about 50% (w/w) to about 90% (w/w), about 50% (w/w) to about 95% (w/w), about 50% (w/w) to about 100% (w/w), about 60% (w/w) to about 70% (w/w), about 60% (w/w) to about 80% (w/w), about 60% (w/w) to about 90% (w/w), about 60% (w/w) to about 95% (w/w), about 60% (w/w) to about 100% (w/w), about 70% (w/w) to about 80% (w/w), about 70% (w/w) to about 90% (w/w), about 70% (w/w) to about 95% (w/w), about 70% (w/w) to about 100% (w/w), about 80% (w/w) to about 90% (w/w), about 80% (w/w) to about 95% (w/w), about 80% (w/w) to about 100% (w/w), about 90% (w/w) to about 95% (w/w), about 90% (w/w) to about 100% (w/w), or about 95% (w/w) to about 100% (w/w)). In some embodiments, HMP is present in the composition at a concentration of about 100% (w/w).

The loss of water alone from a dispersed solution of HMP can lead to the initial polymerization of the pectin. This so-called “gel transition” is associated with a discrete change in the physical properties of the pectin from a viscous liquid to a soft and rubbery gel. The ongoing loss of water from the pectin gel leads to a second discrete step, so called “glass transition,” associated with a change in the physical properties of the pectin from soft and rubbery to hard and brittle. The compositions described herein comprise one or more films in a gel phase or a glass phase. In some embodiments, the film is in a gel phase, and the water content in the film ranges from about 37% (w/w) to about 43% (w/w) (e.g., about 37% (w/w) to about 38% (w/w), about 37% (w/w) to about 39% (w/w), about 37% (w/w) to about 40% (w/w), about 37% (w/w) to about 41% (w/w), about 37% (w/w) to about 42% (w/w), about 37% (w/w) to about 43% (w/w), about 38% (w/w) to about 39% (w/w), about 38% (w/w) to about 40% (w/w), about 38% (w/w) to about 41% (w/w), about 38% (w/w) to about 42% (w/w), about 38% (w/w) to about 43% (w/w), about 39% (w/w) to about 40% (w/w), about 39% (w/w) to about 41% (w/w), about 39% (w/w) to about 42% (w/w), about 39% (w/w) to about 43% (w/w), about 40% (w/w) to about 41% (w/w), about 40% (w/w) to about 42% (w/w), about 40% (w/w) to about 43% (w/w), about 41% (w/w) to about 42% (w/w), about 41% (w/w) to about 43% (w/w), or about 42% (w/w) to about 43% (w/w)). In some embodiments, when in a gel phase, the film is soft, pliable, flexible, rubbery, moldable, or any combination thereof.

In some embodiments, the film is in a glass phase, and the water content in the film ranges from about 9% (w/w) to about 13% (w/w) (e.g., about 9% (w/w) to about 10% (w/w), about 9% (w/w) to about 11% (w/w), about 9% (w/w) to about 12% (w/w), about 10% (w/w) to about 11% (w/w), about 10% (w/w) to about 12% (w/w), about 10% (w/w) to about 13% (w/w), about 11% (w/w) to about 12% (w/w), about 11% (w/w) to about 13% (w/w), or about 12% (w/w) to about 13% (w/w)). In some embodiments, when in a glass phase, the film is rigid.

In some embodiments, the bioadhesive polymer compositions described herein comprise a first film and a second film that are adhered to each other. In some embodiments, the bioadhesive polymer composition comprises first and second films that are conjoined films. In some embodiments, the first and second films become a conjoined film after contacting each other. In some embodiments, the first and second films exhibit a superficial intermingling of their pectin chains that is facilitated by intimate contact and water diffusion, as described in Example 3. In some embodiments, the first and second films comprise a polymer comprising HMP and an initial water content. In some embodiments, the HMP has a degree of methoxylation that is at least about 50%. In some embodiments, the HMP is citrus HMP. In some embodiments, the bioadhesive polymer composition can further include three or more films that are adhered to one another.

In some embodiments, the first film comprises a polymer comprising HMP at a concentration ranging from of about 50% (w/w) to about 100% (w/w) (e.g., about 50% (w/w) to about 60% (w/w), about 50% (w/w) to about 70% (w/w), about 50% (w/w) to about 80% (w/w), about 50% (w/w) to about 90% (w/w), about 50% (w/w) to about 95% (w/w), about 50% (w/w) to about 100% (w/w), about 60% (w/w) to about 70% (w/w), about 60% (w/w) to about 80% (w/w), about 60% (w/w) to about 90% (w/w), about 60% (w/w) to about 95% (w/w), about 60% (w/w) to about 100% (w/w), about 70% (w/w) to about 80% (w/w), about 70% (w/w) to about 90% (w/w), about 70% (w/w) to about 95% (w/w), about 70% (w/w) to about 100% (w/w), about 80% (w/w) to about 90% (w/w), about 80% (w/w) to about 95% (w/w), about 80% (w/w) to about 100% (w/w), about 90% (w/w) to about 95% (w/w), about 90% (w/w) to about 100% (w/w), or about 95% (w/w) to about 100% (w/w)). In some embodiments, HMP is present in the composition at a concentration of about 100% (w/w).

In some embodiments, the second film comprises a polymer comprising HMP at a concentration ranging from of about 50% (w/w) to about 100% (w/w) (e.g., about 50% (w/w) to about 60% (w/w), about 50% (w/w) to about 70% (w/w), about 50% (w/w) to about 80% (w/w), about 50% (w/w) to about 90% (w/w), about 50% (w/w) to about 95% (w/w), about 50% (w/w) to about 100% (w/w), about 60% (w/w) to about 70% (w/w), about 60% (w/w) to about 80% (w/w), about 60% (w/w) to about 90% (w/w), about 60% (w/w) to about 95% (w/w), about 60% (w/w) to about 100% (w/w), about 70% (w/w) to about 80% (w/w), about 70% (w/w) to about 90% (w/w), about 70% (w/w) to about 95% (w/w), about 70% (w/w) to about 100% (w/w), about 80% (w/w) to about 90% (w/w), about 80% (w/w) to about 95% (w/w), about 80% (w/w) to about 100% (w/w), about 90% (w/w) to about 95% (w/w), about 90% (w/w) to about 100% (w/w), or about 95% (w/w) to about 100% (w/w)). In some embodiments, HMP is present in the composition at a concentration of about 100% (w/w).

In some embodiments, the bioadhesive polymer composition comprises one or more films comprising a polymer comprising HMP, water, and carboxymethylcellulose. In some embodiments, one or more films, e.g., one or both of the first and second films, comprise HMP and CMC in a ratio ranging from about 10 to about 1 to about 1 to about 10 by weight (of the composition) (e.g., about 10 to about 1 (w/w), about 9 to about 1 (w/w), about 8 to about 1 (w/w), about 7 to about 1 (w/w), about 6 to about 1 (w/w), about 5 to about 1 (w/w), about 4 to about 1 (w/w), about 3 to about 1 (w/w), about 2 to about 1 (w/w), about 1 to about 1 (w/w), about 1 to about 2 (w/w), about 1 to about 3 (w/w), about 1 to about 4 (w/w), about 1 to about 5 (w/w), about 1 to about 6 (w/w), about 1 to about 7 (w/w), about 1 to about 8 (w/w), about 1 to about 9 (w/w), or about 1 to about 10 (w/w)).

In some embodiments, the bioadhesive polymer composition can further include one or more active agents. For example, the one or more active agents can include one or more anti-bacterial agents or anti-fungal agents. Non-limiting examples of anti-bacterial agents and anti-fungal agents include ciprofloxacin, levofloxacin, doxycycline hyclate, ofloxacin, erythromycin, cefazolin, vancomycin, gentamycin, tobramycin, ceftazidime, gatifloxacin, amphotericin, voriconazole, natamycin, bacitracin, besifloxacin, moxifloxacin, and tobramycin)

In some embodiments, the one or more active agents can include one or more anti-inflammatory agents. Non-limiting examples of anti-inflammatory agents include corticosteroids, loteprednol etabonate, prednisolone acetate, dexamethasone, lifitegrast, cyclosporine, bromfenac, nepafenac, ketorolac, diclofenac, suprofen, and flurbiprofen.

In some embodiments, the one or more active agents can include one or more growth factors, e.g., one or more of transforming growth factor alpha (TGF-a) and TGF-β, tumor necrosis factor-alpha (TNF-α), vascular endothelial growth factor (VEGF), leukemia inhibitory factor (LIF), interleukins such as IL-1 through IL-7, colony-stimulating factors such as macrophage colony-stimulating factor (m-CSF), granulocyte colony-stimulating factor (G-CSF), and granulocyte macrophage colony-stimulating factor (GM-CSF), fibroblast growth factor (FGF), epidermal growth factor (EGF), insulin-like growth factor, connective tissue growth factor (CTGF), hepatocyte growth factor (HGF), angiopoietin-1-4, and platelet-derived growth factor (PDGF). In some embodiments, the one or more films comprise the active agents.

In some embodiments, the one or more active agents can include one or more of heparin, tissue plasminogen activator (tPA), aspirin, ibuprofen, ketoprofen, non-steroidal anti-inflammatory drugs, hormones, cytokines, osteogenic factors, chemotactic factors, proteins and peptides that contain an arginine-glycine-aspartate (“RGD”) motif, analgesics, anesthetics, norepinephrine, epinephrine, phenylpropanolamine, dopamine, metaraminol, methoxamine, ephedrine, propylhexedrine, fibrillar collagen, thrombin, fibrin, a chemotherapy agent, and an immunotherapy agent.

In some embodiments, the concentration of HMP in the polymer of the first film is the same as the concentration of HMP in the polymer of the second film. In some embodiments, the concentration of HMP in the polymer of the first film is different as the concentration of HMP in the polymer of the second film. In some embodiments, the water content in the polymer of the first film is the same as the water content in the polymer of the second film. In some embodiments, the water content in the polymer of the first film is different as the water content in the polymer of the second film. In some embodiments, the first film comprises a polymer comprising HMP and an initial water content at concentrations that are about equal to the concentrations of the polymer that the second film comprises. In some embodiments, the first film comprises a polymer comprising HMP and an initial water content at concentrations that are different to the concentrations of the polymer that the second film comprises.

In some embodiments, the first film is in a gel phase, and the second film is in a glass phase. For example, in some embodiments, the first film comprises a polymer comprising HMP and an initial water content ranging from about 37% to about 43% (w/w). Furthermore, in some embodiments, the second film comprises a polymer comprising HMP and an initial water content ranging from about 9% to about 13% (w/w).

In some embodiments, the first film has an initial water content ranging from about 37% (w/w) to about 43% (w/w) (e.g., about 37% (w/w) to about 38% (w/w), about 37% (w/w) to about 39% (w/w), about 37% (w/w) to about 40% (w/w), about 37% (w/w) to about 41% (w/w), about 37% (w/w) to about 42% (w/w), about 37% (w/w) to about 43% (w/w), about 38% (w/w) to about 39% (w/w), about 38% (w/w) to about 40% (w/w), about 38% (w/w) to about 41% (w/w), about 38% (w/w) to about 42% (w/w), about 38% (w/w) to about 43% (w/w), about 39% (w/w) to about 40% (w/w), about 39% (w/w) to about 41% (w/w), about 39% (w/w) to about 42% (w/w), about 39% (w/w) to about 43% (w/w), about 40% (w/w) to about 41% (w/w), about 40% (w/w) to about 42% (w/w), about 40% (w/w) to about 43% (w/w), about 41% (w/w) to about 42% (w/w), about 41% (w/w) to about 43% (w/w), or about 42% (w/w) to about 43% (w/w)). In some embodiments, the second film has an initial water content of about 40% (w/w).

In some embodiments, the second film has an initial water content ranging from about 9% (w/w) to about 13% (w/w) (e.g., about 9% (w/w) to about 10% (w/w), about 9% (w/w) to about 11% (w/w), about 9% (w/w) to about 12% (w/w), about 10% (w/w) to about 11% (w/w), about 10% (w/w) to about 12% (w/w), about 10% (w/w) to about 13% (w/w), about 11% (w/w) to about 12% (w/w), about 11% (w/w) to about 13% (w/w), or about 12% (w/w) to about 13% (w/w)). In some embodiments, the second film has an initial water content of about 10% (w/w).

In some embodiments, the first film is in a gel phase, and the second film is in a gel phase. In some embodiments, the first film is in a gel phase, and the second film is in a glass phase. In some embodiments, the conjoined films are both in a glass phase (e.g., after compression and dehydration).

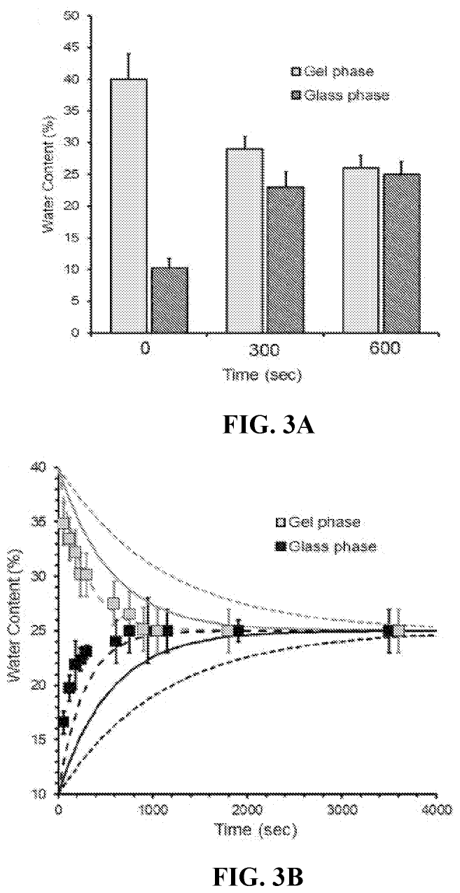

As used herein, the term “initial water content” of the first and second films is a water content before and during an initial period of time after the first and second films are adhered to each other. For example, in some embodiments, the first and second films have an initial water content prior to being adhered to each other and reach a water equilibrium within about 10 minutes of being in contact with each other, as shown in FIG. 3A. In some embodiments, after reaching water equilibrium, the first and second conjoined films reach a final water content. As used herein, the term “final water content” of the first and second films is a water content present in each film after the initial period of time has lapsed (e.g., after reaching water equilibrium).

In some embodiments, the initial period of time is about 10 minutes at most (e.g., about 1 second (s) to about 5 s, about 5 s to about 10 s, about 10 s to about 20 s, about 20 s to about 30 s, about 30 s to about 40 s, about 40 s to about 50 s, about 50 s to about 1 minute (min.), about 1 min. to about 2 min., about 2 min. to about 3 min., about 3 min. to about 4 min., about 4 min. to about 5 min., about 5 min. to about 6 min., about 6 min. to about 7 min., about 7 min. to about 8 min., about 8 min. to about 9 min., about 9 min. to about 10 min., about 1 s to about 30 s, about 1 s to about 1 min., about 1 s to about 5 min., about 1 s to about 10 min., about 30 s to about 1 min., about 30 s to about 5 min., about 30 s to about 10 min., about 30 s to about 1 min., about 1 min. to about 3 min., about 1 min. to about 4 min., about 1 min. to about 5 min., about 1 min. to about 6 min., about 1 min. to about 7 min., about 1 min. to about 8 min., about 1 min. to about 9 min., about 1 min. to about 10 min., or about 5 min. to about 10 mins.)

In some embodiments, once adhered to each other, the first and second films exhibit interdiffusion of water. For example, the first and second films have an initial water content that is different to a final water content. In some embodiments, the final water content in the conjoined films ranges from about 20% (w/w) to about 30% (w/w) (e.g., about 20% (w/w) to about 30% (w/w), about 21% (w/w) to about 30% (w/w), about 22% (w/w) to about 30% (w/w), about 23% (w/w) to about 30% (w/w), about 24% (w/w) to about 30% (w/w), about 25% (w/w) to about 30% (w/w), about 26% (w/w) to about 30% (w/w), about 27% (w/w) to about 30% (w/w), about 28% (w/w) to about 30% (w/w), about 29% (w/w) to about 30% (w/w), about 20% (w/w) to about 25% (w/w), or about 25% (w/w) to about 30% (w/w)). In some embodiments, the final water content in the conjoined films is about 25% (w/w). In some embodiments, a diffusion coefficient of water through the first and second films is about 2.5-fold to about 5-fold (e.g., about 2.5-fold to about 3-fold, about 3-fold to about 4-fold, about 3-fold to about 5-fold, or about 4-fold to about 5-fold) slower than a self-diffusion coefficient of water.

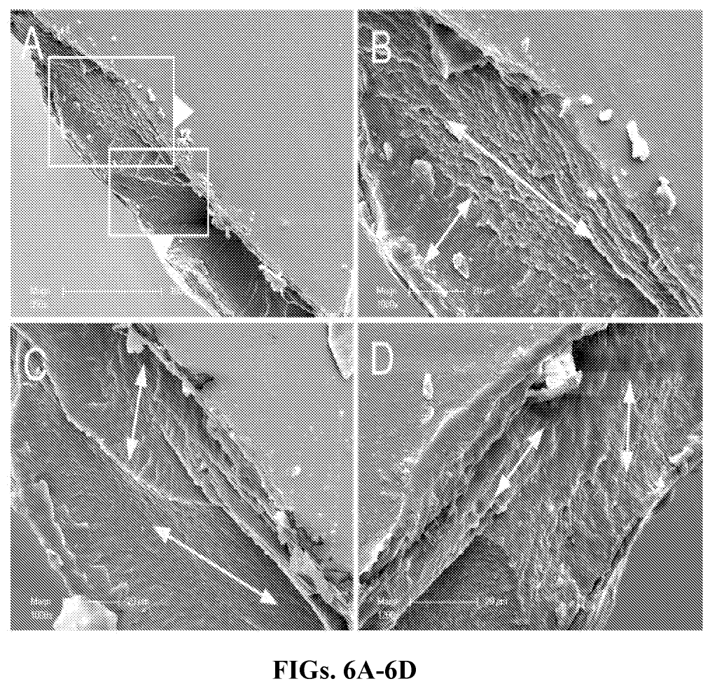

As shown in FIG. 6 and as described in Example 2, the physical interface of the conjoined films was evaluated by scanning electron microscopy. In some embodiments, the conjoined films have a cross-grain texture at physical interface between the first and second films. In some embodiments, the water content at the physical interface between the first and second films differs from the water content elsewhere in the bulk of the first and second films.

As described in Example 2, the adhesivity of the first film to a second film can vary depending on the phase of each film. For example, in some embodiments, the adhesivity of a first gel phase film to a second glass phase film is greater than the adhesivity of a first glass phase film to a second glass phase film. In some embodiments, the adhesivity of a first gel phase film to a second glass phase film is about the same or greater than the adhesivity of a first gel phase film to a second gel phase film. In some embodiments, the adhesivity of the first and second films (e.g., a first gel phase film and a second glass phase film) ranges from about 22 newton (N) to about 28 N (e.g., about 22 N to about 23 N, about 22 N to about 24 N, about 22 N to about 25 N, about 22 N to about 26 N, about 22 N to about 27 N, about 22 N to about 28 N, about 23 N to about 24 N, about 23 N to about 25 N, about 23 N to about 26 N, about 23 N to about 27 N, about 23 N to about 28 N, about 24 N to about 25 N, about 24 N to about 26 N, about 24 N to about 27 N, about 24 N to about 28 N, about 25 N to about 26 N, about 25 N to about 27 N, about 25 N to about 28 N, about 26 N to about 27 N, about 26 N to about 28 N, or about 27 N to about 28 N). In some embodiments, the adhesivity of the first and second films (e.g., a first gel phase film and a second glass phase film) is about 25 N.

In some embodiments, the adhesivity of the first and second films (e.g., a first gel phase film and a second gel phase film) ranges from about 27 newton (N) to about 33 N (e.g., about 27 N to about 28 N, about 27 N to about 29 N, about 27 N to about 30 N, about 27 N to about 31 N, about 27 N to about 32 N, about 27 N to about 33 N, about 28 N to about 29 N, about 28 N to about 30 N, about 28 N to about 31 N, about 28 N to about 32 N, about 28 N to about 33 N, about 29 N to about 30 N, about 29 N to about 31 N, about 29 N to about 32 N, about 29 N to about 33 N, about 30 N to about 31 N, about 30 N to about 32 N, about 30 N to about 33 N, about 31 N to about 32 N, about 31 N to about 33 N, or about 32 N to about 33 N). In some embodiments, the adhesivity of the first and second films (e.g., a first gel phase film and a second gel phase film) is about 30 N.

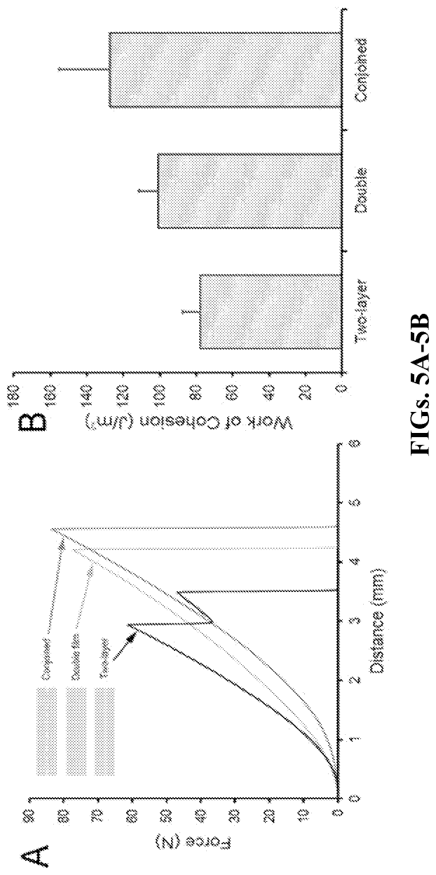

In some embodiments, once adhered to each other, the first and second films exhibit interdiffusion of HMP. This polymer-polymer pectin interdiffusion exhibited by conjoined films can lead to an increase in work of cohesion and extensibility, as shown in FIGS. 5A-B and as described in Example 2. In some embodiments, the conjoined films have an extensibility ranging from between about 30% to about 40% (e.g., about 30% to about 31%, about 30% to about 32%, about 30% to about 33%, about 30% to about 34%, about 30% to about 35%, about 30% to about 36%, about 30% to about 37%, about 30% to about 38%, about 30% to about 39%, about 30% to about 35%, or about 35% to about 40%) greater than a double-thickness film or a stacked, two-layer film. In some embodiments, the conjoined films have an extensibility that is about 30% greater than a double-thickness film or a stacked, two-layer film. In some embodiments, the conjoined films have an extensibility that is about 39% greater than a double-thickness film or a stacked, two-layer film.

In some embodiments, the conjoined films have a work of cohesion ranging from about 100 J/m² to about 150 J/m² (e.g., about 100 J/m² to about 110 J/m², about 100 J/m² to about 120 J/m², about 100 J/m² to about 130 J/m², about 100 J/m² to about 140 J/m², about 100 J/m² to about 149 J/m², about 110 J/m² to about 120 J/m², about 110 J/m² to about 130 J/m², about 110 J/m² to about 140 J/m², about 110 J/m² to about 150 J/m², about 120 J/m² to about 130 J/m², about 120 J/m² to about 140 J/m², about 120 J/m² to about 150 J/m², about 130 J/m² to about 140 J/m², about 130 J/m² to about 150 J/m², or about 140 J/m² to about 150 J/m²). In some embodiments, the conjoined films have a work of cohesion that is about 120 J/m².

In some embodiments, the bioadhesive polymer composition comprises a film having a thickness ranging from about 40 μm to about 200 μm (e.g., about 40 μm to about 50 μm, about 40 μm to about 60 μm, about 40 μm to about 70 μm, about 40 μm to about 80 μm, about 40 μm to about 90 μm, about 40 μm to about 100 μm, about 40 μm to about 110 μm, about 40 μm to about 120 μm, about 40 μm to about 130 μm, about 40 μm to about 140 μm, about 40 μm to about 150 μm, about 40 μm to about 160 μm, about 40 μm to about 170 μm, about 40 μm to about 180 μm, about 40 μm to about 190 μm, about 40 μm to about 199 μm, about 50 μm to about 60 μm, about 50 μm to about 70 μm, about 50 μm to about 80 μm, about 50 μm to about 90 μm, about 50 μm to about 100 μm, about 50 μm to about 110 μm, about 50 μm to about 120 μm, about 50 μm to about 130 μm, about 50 μm to about 140 μm, about 50 μm to about 150 μm, about 50 μm to about 160 μm, about 50 μm to about 170 μm, about 50 μm to about 180 μm, about 50 μm to about 190 μm, about 50 μm to about 200 μm, about 60 μm to about 70 μm, about 60 μm to about 80 μm, about 60 μm to about 90 μm, about 60 μm to about 100 μm, about 60 μm to about 110 μm, about 60 μm to about 120 μm, about 60 μm to about 130 μm, about 60 μm to about 140 μm, about 60 μm to about 150 μm, about 60 μm to about 160 μm, about 60 μm to about 170 μm, about 60 μm to about 180 μm, about 60 μm to about 190 μm, about 60 μm to about 200 μm, about 70 μm to about 80 μm, about 70 μm to about 90 μm, about 70 μm to about 100 μm, about 70 μm to about 110 μm, about 70 μm to about 120 μm, about 70 μm to about 130 μm, about 70 μm to about 140 μm, about 70 μm to about 150 μm, about 70 μm to about 160 μm, about 70 μm to about 170 μm, about 70 μm to about 180 μm, about 70 μm to about 190 μm, about 70 μm to about 200 μm, about 80 μm to about 90 μm, about 80 μm to about 100 μm, about 80 μm to about 110 μm, about 80 μm to about 120 μm, about 80 μm to about 130 μm, about 80 μm to about 140 μm, about 80 μm to about 150 μm, about 80 μm to about 160 μm, about 80 μm to about 170 μm, about 80 μm to about 180 μm, about 80 μm to about 190 μm, or about 80 μm to about 200 μm). In some embodiments, the bioadhesive polymer composition comprises a film having a thickness of about 80 μm.

In some embodiments, the bioadhesive polymer composition comprises conjoined films where a first film has a thickness that is about equal to a thickness of a second film. In some embodiments, the bioadhesive polymer composition comprises conjoined films where the first film has a thickness that is different from a thickness of the second film. For example, in some embodiments, the thickness of the first film (e.g., a film in a gel phase) is about 62 μm. Furthermore, in some embodiments, the thickness of the second film (e.g., a film in a glass phase) is about 40 μm.

In some embodiments, the bioadhesive polymer composition comprises conjoined films where a first film has a reduced bioadhesion compared to the second film, or the second film has a reduced bioadhesion compared to the first film. The result is a film that is more bioadhesive on one side than the other, which is an important feature when using the films to reduce the risk of tissue adhesions (e.g., post-surgical tissue adhesions such as post-surgical pleural/serosal adhesions). In some embodiments, a surface of a first film of the conjoined films has a bioadhesive that includes at least about 50% pectin (e.g., about 50% to about 60%, about 50% to about 70%, about 50% to about 80%, about 50% to about 90%, or about 50% to about 100%). In some embodiments, a surface of a second film of the conjoined films has a non-bioadhesive (or a reduced bioadhesive) side that includes less than about 15% pectin (e.g., about 15% to about 14%, about 15% to about 13% about 15% to about 12%, about 15% to about 11%, about 15% to about 10%, about 15% to about 9%, about 15% to about 8%, about 15% to about 7%, about 15% to about 6%, about 15% to about 5%, about 15% to about 4%, about 15% to about 3%, about 15% to about 2%, about 15% to about 1%, or about 15% to about 0%) and greater than about 15% CMC or other non-bioadhesive, biodegradable cellulose-related polymer and/or silicone. In some embodiments, the non-bioadhesive side may include a non-biodegradable, but biologically inert, material.

In some embodiments, the bioadhesive polymer composition further includes a pharmaceutically acceptable carrier. As used herein, the expression “pharmaceutically acceptable carrier” refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation and is compatible with administration to a subject, for example a human. It must also be suitable for use in contact with any tissues or organs with which it may come in contact, meaning that it must not carry a risk of toxicity, irritation, allergic response, immunogenicity, or any other complication that excessively outweighs its therapeutic benefits. Examples of pharmaceutically acceptable carriers include, but are not limited to, a solvent or dispersing medium containing, for example, water, pH buffered solutions (e.g., phosphate buffered saline (PBS), HEPES, TES, MOPS, etc.), isotonic saline, Ringer's solution, polyol (for example, glycerol, propylene glycol, liquid polyethylene glycol, and the like), alginic acid, ethyl alcohol, and suitable mixtures thereof. In some embodiments, the pharmaceutically acceptable carrier can be a pH buffered solution (e.g. PBS).

In some embodiments, the pharmaceutically acceptable carrier is a topical carrier. In some embodiments, the bioadhesive polymer composition is formulated for topical use. In some embodiments, the composition is topically administered to a tissue (e.g., an ocular tissue) of a patient. In some embodiments, the composition can be applied to a tissue (e.g., an ocular tissue) to seal an ocular injury (e.g., an ocular incision).

### Methods of Preparation

Certain embodiments of the disclosure include methods of preparing a conjoined bioadhesive polymer composition. In some embodiments, the methods include providing a first film and a second film, each film comprising a polymer comprising HMP and water. For example, the first and second film can include any of compositions described elsewhere herein.

In some embodiments, the first and second films can be prepared by methods described in Example 1. For example, the first and second films can be prepared by gradually dissolving the polymer (e.g., HMP) powder in water at a temperature of about 25° C. For example, the HMP powder can be dissolved by a step-wise increase in added water to avoid undissolved powder. In some embodiments, the HMP powder is dissolved to a mixing concentration of about 3% (w/w) (e.g., about 3 g of HMP powder is dissolved in about 100 g of water). In some embodiments, the HMP powder is dissolved to a mixing concentration of about 1% (w/w) to about 3% (w/w).

In some embodiments, no exogenous heat is used to dissolve the polymer (e.g., HMP). Next, the methods include mixing at a high shear (e.g., mixing at about 10,000 revolutions per minute (RPM)) to achieve fluidization and complete dissolution of HMP. Next, the dissolved polymer solution can be transferred to one or more molds and cured (e.g., via a controlled evaporation of water) in an environment having a controlled humidity. In some embodiments, the controlled humidity environment is a low humidity environment (e.g., less than about 20% relative humidity) that enables the progressive loss of water content of the polymer solution in the mold. In some embodiments, the thickness of the films can be controlled by the dimensions of the mold in which the polymer solution is poured into and cured.

In some embodiments, the methods include progressively evaporating the water in each film until the first film is in a gel phase and the second film is in a glass phase. In some embodiments, the first film has a water content ranging from about 37% to about 43% (w/w) once it is in the gel phase. In some embodiments, the second film has a water content ranging from about 9% to about 13% (w/w) once it is in the glass phase.

Next, the methods can include contacting the first film (e.g., in a gel phase) and the second film (e.g., in a glass phase) with each other and compressing both films. In some embodiments, the first and second films are compressed with a force of about 10 N to about 80 N (e.g., about 10 N to about 20 N, about 10 N to about 30 N, about 10 N to about 40 N, about 10 N to about 50 N, about 10 N to about 60 N, about 10 N to about 70 N, about 10 N to about 79 N, about 20 N to about 30 N, about 20 N to about 40 N, about 20 N to about 50 N, about 20 N to about 60 N, about 20 N to about 70 N, or about 20 N to about 80 N). In some embodiments, the first and second films are compressed with a force of about 20 N. In some embodiments, the first and second films are compressed for about 10 seconds to about 12 hours (e.g., about 10 seconds to about 30 seconds, about 10 seconds to about 1 min., about 10 seconds to about 2 min., about 10 seconds to about 5 min., about 10 seconds to about 10 min., about 10 seconds to about 60 min., about 10 seconds to about 5 hours, about 10 seconds to about 10 hours, about 10 seconds to about 12 hours, about 1 min. to about 5 min., about 1 min. to about 10 min., about 10 min. to about 60 min., about 10 min. to about 5 hours, or about 10 min. to about 12 hours).

In some embodiments, the first and second films are adhered to one another after the compression step. In some embodiments, the first and second films exhibit interdiffusion of water and pectin polymer upon contact with each other. In some embodiments, the water content of the first and second films changes after the first film and second film contact each other. In some embodiments, the water content (e.g., water equilibrium concentration) in the conjoined films ranges from about 20% to about 30% (w/w) after the compressing step. In some embodiments, the water equilibrium concentration in the conjoined films is reached after about 10 minutes at most (e.g., about 5 seconds to 10 seconds, about 10 seconds to about 30 seconds, about 30 seconds to about 1 min., about 1 min. to about 5 min., or about 5 min. to about 10 min.) after the compressing step.

### Methods of Treatment

In some embodiments, the compositions (e.g., conjoined films) of the disclosure may facilitate the sealing of a visceral organ (e.g., lung, liver, bowel, heart, or any combination thereof) wound of a subject upon application and adhesion. In some embodiments, the compositions (e.g., conjoined films) of the disclosure may facilitate the sealing of a wound of a mesothelial tissue of a subject upon application and adhesion. In some embodiments, the compositions (e.g., conjoined films) of the disclosure may facilitate the sealing of a wound of a tissue having a glycocalyx surface (e.g., lung, liver, bowel, heart, eye, or any combination thereof) upon application and adhesion. In some embodiments, the HMP chains of the compositions exhibit entanglement with the glycocalyx of a tissue upon contact, thereby resulting in adhesion of the compositions to the tissue.

In some embodiments, the compositions of the disclosure may facilitate the sealing and/or treating of an ocular injury in an eye of a subject upon application and adhesion. For example, the compositions of the disclosure can be contacted with an ocular surface, can subsequently adhere to the ocular surface, and can seal an ocular injury. In some embodiments, the methods of sealing and/or treating an ocular injury include providing a bioadhesive polymer composition that is a single film. In some embodiments, the methods of sealing and/or treating an ocular injury include providing a bioadhesive polymer composition that includes two or more films adhered to each other (e.g., conjoined films).

In some embodiments, the methods can include providing a bioadhesive film comprising a polymer comprising HMP and water (e.g., any of the bioadhesive polymer compositions of the disclosure). Next, the methods can include contacting the eye of the subject with the bioadhesive polymer film. In some embodiments, the methods include applying pressure to the bioadhesive polymer film once it comes in contact with the eye. In some embodiments, the pressure applied to the bioadhesive polymer film is a light pressure (e.g., about the same amount of pressure applied when adhering a piece of Scotch® tape to a paper). In some embodiments, the methods do not require the step of applying pressure to the bioadhesive film once it comes in contact with the eye. In some embodiments, the pressure is applied for about 10 seconds (s) or less (e.g., about 1 s to about 2 s, about 1 s to about 3 s, about 1 s to about 4 s, about 1 s to about 5 s, about 1 s to about 6 s, about 1 s to about 7 s, about 1 s to about 8 s, about 1 s to about 9 s, about 1 s to about 10 s, or about 5 s to about 10 s). In some embodiments, the bioadhesive film reaches 80% of maximal adhesion to the eye within at least about 5 seconds of contact, thereby sealing the ocular injury in the eye of the subject. In some embodiments, the bioadhesive film adheres to the eye of the subject with an adhesion strength of at least about 3 N.

In some embodiments, the bioadhesive film is flexible, translucent, bioabsorbable, fracture-resistant, or any combination thereof. In some embodiments, the bioadhesive film exhibits minimal optical distortion. In some embodiments, after the bioadhesive film seals the ocular injury, the eye is resistant to a pressure fluctuation ranging from about 50 mmHg to about 280 mmHg (e.g., about 50 mmHg to about 60 mmHg, about 50 mmHg to about 70 mmHg, about 50 mmHg to about 80 mmHg, about 50 mmHg to about 90 mmHg, about 50 mmHg to about 100 mmHg, about 50 mmHg to about 150 mmHg, about 50 mmHg to about 200 mmHg, about 50 mmHg to about 250 mmHg, about 50 mmHg to about 279 mmHg, about 100 mmHg to about 150 mmHg, about 100 mmHg to about 200 mmHg, about 100 mmHg to about 250 mmHg, about 100 mmHg to about 280 mmHg, about 150 mmHg to about 200 mmHg, about 150 mmHg to about 250 mmHg, about 150 mmHg to about 280 mmHg, about 200 mmHg to about 250 mmHg, about 200 mmHg to about 280 mmHg, or about 250 mmHg to about 280 mmHg). In some embodiments, after the bioadhesive film seals the ocular injury, the eye is resistant to a pressure fluctuation of about 200 mmHg.

In some embodiments, the bioadhesive film is used to prevent fluid leakage after cataract surgery. In some embodiments, the bioadhesive film remains localized over the incision, injury, and/or laceration to seal the wound and form a surface barrier. In some embodiments, the bioadhesive film is a biocompatible and adhesive sealant on the ocular surface.

### Ocular Injuries

The present disclosure presents methods and compositions for treating ocular injuries (e.g., ocular surface injuries) in an eye of a subject. In some embodiments, the ocular injury is a corneal injury. In some embodiments, the ocular injury is a corneal incision. In some embodiments, the ocular injury is an injury or trauma resulting from an ocular surgery. In some embodiments, the ocular surgery is cataract surgery. In some embodiments, the compositions of the disclosure are used in post-surgical care. For example, the compositions can be administered to a patient after an ocular surgery to delivery a therapeutic agent (e.g., an anti-inflammatory agent or an antibiotic) that can be prescribed to minimize recovery time, prevent and/or treat inflammation caused by the surgical procedure, prevent and/or treat an ocular infection caused by the surgical procedure, or any combination thereof.

Ocular surface injuries can include conjunctival laceration, corneal perforation, scleral perforation, incisions due to ocular surgery (e.g., cataract surgery) or any combination thereof. In some embodiments, the ocular surface injury is a corneal or scleral injury. Conjunctival laceration can occur following blunt or penetrating trauma. Conjunctival lacerations can be associated with chemosis and subconjunctival hemorrhage. In such cases, it is important to rule out underlying scleral perforation. The fundus should be examined for any retinal tear or intraocular foreign body. An ultrasound can be done for the posterior segment evaluation.

Corneal lacerations and perforations represent approximately 1 in 10 of ocular traumatic injuries presenting in an emergency medical setting. Corneal lacerations and perforations can include partial thickness lacerations and full thickness lacerations. In addition, adnexal injuries, scleral perforation, or a combination thereof can be involved with corneal laceration and perforations. The standard of care for a corneal perforation include the removal of any contaminants in the wound area, repair of the tear, and maintenance of the watertight integrity of the ocular globe. Corneal perforation can also be associated with or caused by insertion of a foreign body. In some embodiments, the corneal injury is a corneal full-thickness laceration or a corneal full-thickness perforation. In some embodiments, the ocular surface injury is a full-thickness laceration or a full-thickness perforation. In some embodiments, the ocular surface injury is a full-thickness laceration or surgical incision or a full-thickness perforation. For example, the majority of ocular surgeries that require entry into the eye (e.g., cataract surgery) involve a full-thickness incision through the cornea or sclera. Current management protocols for full thickness lacerations including scleral wounds often require the use of sutures.

The compositions of the disclosure can be used to treat ocular incisions or cuts or injuries having a length of less than about 1 mm to about 10 mm. In some embodiments, the compositions of the present disclosure can be used in the closure of full-thickness ocular defects and lacerations and in controlled and long-term drug elution. In some embodiments, indications can include post-operative applications of the biomaterial for drug elution in addition of closure of corneal ulcers, defects and perforations caused by a wide array of insults. The compositions of the disclosure can be applied both under “normal” (e.g., in-the-office or operating room) settings, or under emergency “in-in-field” settings. Various providers, physicians, and, in select cases, physician assistants and paramedics (e.g., in the combat theater) can apply the compositions described herein to seal the eye and elute drug(s) to heal defects. The compositions described herein can circumvent many cases of transplants and patch grafts for corneal melts and defects.

### EXAMPLES

Certain embodiments of the present disclosure are further described in the following examples, which do not limit the scope of any embodiments described in the claims.

### Example 1—Synthesis and Physical Characterization of Pectin Films Pectin

Citrus pectins were obtained from a commercial source (Cargill, Minneapolis, MN, USA). Briefly, the proportion of galacturonic acid residues in the methyl ester form determined the degree of methoxylation. High-methoxyl pectins (HMP) were defined as those pectin polymers with a greater than about 50% degree of methoxylation (Mean=66±9%). The mean molecular weight was about 265 kD, intrinsic viscosity was 621 mL/g, and the polydispersity index 1.84. The pectin powder was stored in low humidity at 25° C.

### Pectin Dissolution in Water

The pectin powder was dissolved at 25° C. by a gradual increase in added water to avoid undissolved powder as previously described. Swelling and softening of the particles was followed by fluidization; complete dissolution of the pectin was achieved by a high-shear 10,000 rpm rotor-stator mixer (L5M-A Model, Silverson®, East Longmeadow, MA, USA). The dissolved pectin (3% w/w) was poured into standard molds for further studies. The thickness of the films (h) varied with water content and amount of pectin: 40±0.4 um thick in glass phase (12% water content) and 62±0.6 urn thick in gel phase (40% water content).

### Humidification Chamber

A custom designed 5.7 L translucent polycarbonate humidification chamber was used to maintain stable humidity during prolonged testing. Humidification was produced by an ultrasonic humidifier or manual aerosol device. The chamber was monitored by wireless (Bluetooth) hygrometer and thermometer sensors (SensorPush®, Brooklyn, NY, USA). The data recording device was maintained within the humidification chamber throughout each experiment. The instrument was designed to be compatible with the AT-XT plus instrument (Stable Micro Systems, Godalming, Surrey, UK).

### Adhesion Testing

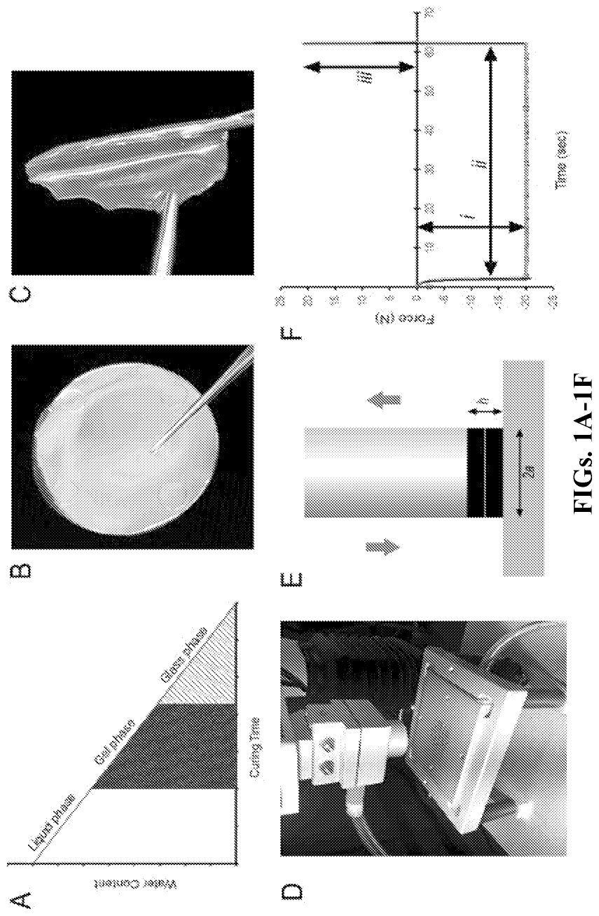

Polymer-polymer adhesion experiments were performed with a force-calibrated custom fixture designed for the TA-XT plus with 50 kg load cell (Stable Micro Systems, Godalming, Surrey, UK, FIG. 1). The fixture was composed of a 30 mm diameter flat-ended cylindrical probe and a flat fixture surface designed with vacuum fixation. The films were attached to polychloroprene mount using proprietary 3M adhesive (3M™, St. Paul, MN, USA) or cyanoacrylate adhesive (VetBone™, 3M, St. Paul, MN, USA). The cylindrical probe compressed the two polymers followed by the separation of the probe from the surface by an applied tensile load. The radius of contact was defined as the radius of the probe (a) and the thickness of the contacted polymers was h. The pre- and post-experimental geometries were nearly equivalent for large values of a/h with minimal evidence of edge effects. Probe velocity, compression force and distance were recorded at 500 pps. A minimum of N=10 films per data point were tested.

### Fracture Mechanics

To determine the fracture mechanics of the pectin, the biopolymers were subjected to a controlled uniaxial load normal to the plane of the polymer film. Briefly, a 5 mm stainless steel spherical probe was mounted to a 50 kg load cell and positioned centrally over the biopolymer. The probe descended at a velocity of 1 mm/s until contact with the film. At a 0.049 N trigger force, then a probe velocity increased to 2 mm/s until fracture. The fracture force and distance were recorded at 500 pps. Burst strength was defined as the peak force required for film fracture (N). The distance the probe traveled between polymer contact (detection at 1 N) and film fracture was defined as extensibility (mm). The slope of the initial linear portion of the burst curve was defined as stiffness (N/mm).

### Scanning Electron Microscopy

After coating with 20-25 A gold in an argon atmosphere, the pectin films were imaged using a Philips XL30 ESEM scanning electron microscope (Philips, Eindhoven, The Netherlands) at 15 keV and 21 μA. Images were obtained using a eucentric sample holder using standardized automation.

### Statistical Analysis

The statistical analysis was based on measurements in at least three different samples. The unpaired Student's t test for samples of unequal variances was used to calculate statistical significance. The data was expressed as mean±one standard deviation (SD). The significance level for the sample distribution was defined as p<0.01.

### Example 2—Mechanical Properties of Pectin Films Gel and Glass Phase Film Adhesion

Pectin films were cured in a controlled humidity microenvironment. In low humidity, the progressive loss of water content (W_{c}) of the pectin resulted in the transition of the liquid pectin into gel phase (soft, rubbery) and subsequently glass phase (hard, brittle) films (FIGS. 1A-1F).

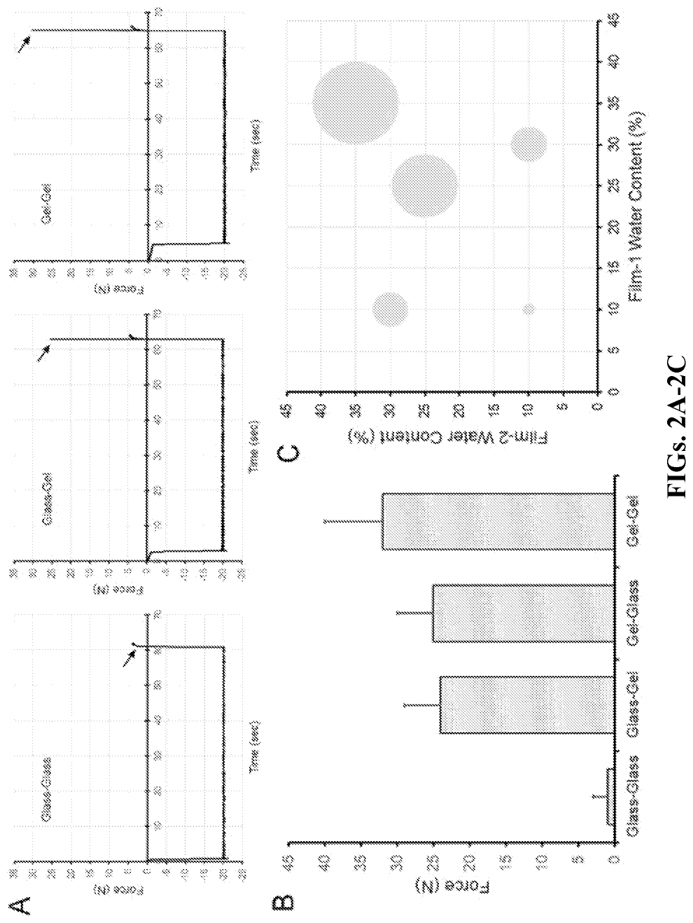

The adhesivity of two glass phase films (glass-glass) was less than 5 newton (N) (FIG. 2A). The adhesivity of one glass phase and one gel phase films (glass-gel) was 25±3 N (FIG. 2A). The adhesivity of two gel phase films (gel-gel) was 30±3 N (FIG. 2A). The adhesive strength of glass-glass films was significantly less than the adhesive strength of glass-gel or gel-gel films (p<0.0001) (FIG. 2B, 2C).

### Polymer-Polymer Water Interdiffusion

To assess the potential diffusion of water between a gel phase film (40±3% W_{c}) and a glass phase film (11±2% W_{c}), the films were compressed, then separated at various time intervals after initial contact. The water content was assessed by film weight. The standard glass and gel phase films reached a water equilibration within 10 minutes (FIG. 3A). Since the diffusivity of water through the pectin medium is unknown, the empirical diffusion data was plotted against theoretical plots reflecting a range of diffusion coefficients at 25° C. The diffusion coefficient of water through the pectin films was estimated to be 2.5- to 5-fold slower than the self-diffusion coefficient of water (FIG. 3B).

### Compression and Development Time

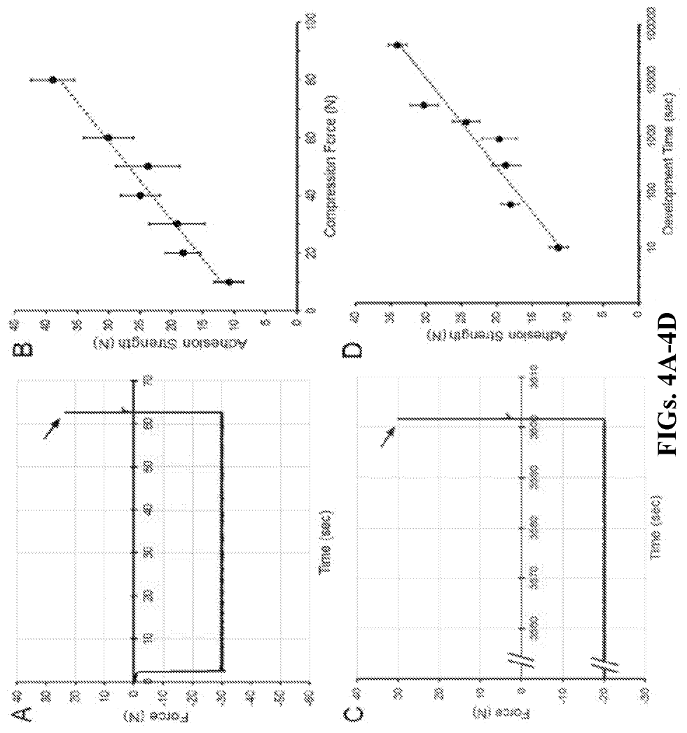

The effect of polymer-polymer compression on adhesivity was assessed using an adhesion assay with two gel phase polymers. The compressive force, varied from 10 N to 80 N, was applied for 60 s (FIG. 4A, 4B). The adhesive strength increased with increasing compression (R²=0.956) (FIG. 4B). To study the influence of development time on polymer-polymer adhesion, a controlled humidity microenvironment was used to maintain polymer water content over time. Gel phase films were compressed with a force of 20 N for time periods varying from 10 s to 12 h (FIG. 4C, 4D). The compression time and adhesive strength demonstrated a logarithmic relationship (R²=0.913) with most of the adhesive force developing within minutes of contact (FIG. 4D).

### Polymer-Polymer Pectin Interdiffusion

The fracture mechanics of three types of films were studied—each with comparable pectin (88±1 mg) and water (13±1%) content: (1) conjoined films produced by compression (10 s at 10 N) of two gel phase films followed by curing to glass phase; (2) double-thickness films produced by high-shear mixing and curing to glass phase; (3) two-layer films produced by stacking single-layer glass phase films (FIGS. 5A and 5B). Fracture mechanics were assessed using a constant velocity uniaxial load applied normal to the plane of the polymer film until fracture. The conjoined and double-thickness films produced a single fracture peak, but the two-layer films demonstrated a bimodal fracture pattern suggesting the independent fracture of the two stacked films (FIG. 5A). Notably, the burst strength of conjoined films (82±14 N), double-thickness films (71±12 N) and two stacked (two-layer) films (70±16 N) was similar (p>0.01). In contrast, the conjoined films demonstrated decreased stiffness (conjoined 19.4±4.1 N/mm; double, 28.3±1.6 N/mm; two-layer, 26.3±0.9 N/mm) and increased extensibility (conjoined, 4.3±0.5 mm; double, 3.1±0.2 mm; two-layer, 3.3±0.3 mm) (p<0.01). Consistent with these findings, the area under the fracture curve, reflecting the work of cohesion, was 30% greater in the conjoined films than the double-thickness or two-layer films (FIG. 5B) (p<0.01).

### Scanning Electron Microscopy

To investigate the structural basis for these cohesive properties, the physical interface of the conjoined films was evaluated by SEM. Conjoined films were produced by compression (60 s at 20 N) followed by dehydration to the glass phase. The films were sharply bisected and examined by SEM (FIGS. 6A-6D). The interface between the two conjoined films suggested a cross-grain texture (FIGS. 6B-6D).

### Example 3—Conjoined Films Exhibit Greater Cohesive Strength

A promising approach to regulate pectin biostability is the blending of pectin films. To investigate the development of conjoined films, the physical properties of high-methoxyl pectin polymer-polymer (homopolymer) interactions at the adhesive interface were examined. Several empirical observations were made. (1) Water content greater than 10-13% (w/w) was required for pectin polymer-polymer adhesion. (2) Adhesion occurred rapidly (minutes) above a minimum compression threshold. (3) Polymer-polymer adhesion produced a conjoined film with distinctive cohesion properties. (4) SEM demonstrated evidence of superficial pectin bridging and cross-grain adhesion of the conjoined polymers. It was concluded that pectin polymer-polymer adhesion involves a process of superficial intermingling of pectin chains that is facilitated by intimate contact and water diffusion.

The experimental conditions were designed to simulate the adhesive interactions encountered in biomedical applications; namely, the polymer interactions were studied in air and with a water content restricted to glass and gel phase polymers. There are intriguing conceptual issues that remain undefined. First, the water content at the polymer interface may differ from the water content of the bulk film. Whereas this difference may influence theoretical issues of surface contact, polymer deformation and chain mobility, the reproducibility of the adhesion studies suggests that these issues may be of limited practical significance. Second, pectin is a negatively charged polyelectrolyte potentially influenced by salt concentration. The study was restricted to water and a meticulously maintained humidified microenvironment. Although the influence of ions on pectin adhesion is relevant and should be considered in future studies, the recent studies indicate that phase state is the dominant predictor of both physical properties and polymer-polymer adhesion.

The distinctive feature of the initial contact or wetting phase of polymer-polymer interaction is the rapidity of both adhesion and de-adhesion. Adhesion occurred within seconds of contact; similarly, de-adhesion occurred suddenly—producing the near-vertical de-adhesion curve observed in the tensile strength assay. This water-dependent adhesion likely involves hydrogen bonding, the hydrophobic interaction between methyl groups and electrostatic forces between polymer chains. Although the SEM studies demonstrated evidence of pectin chains traversing the interface between conjoined films, these images can be interpreted as reflecting the superficial intermingling, rather than the substantive interpenetration, of pectin chains. It is anticipated that more substantial chain interpenetration would produce effacement of the polymer interface as well as a more protracted debonding curve.

The adhesion between two gel-phase pectin polymers not only occurred rapidly, but the strength of adhesion increased with increasing compression. The progressive development of adhesive strength over time suggests the consolidation of the physico-chemical interactions involved in water-dependent adhesion. Similarly, the compression forces may have contributed to inter-polymer consolidation by reducing the distance between interacting polymers.

An unexpected finding was the cohesive strength of the conjoined films. Two compressed gel-phase (conjoined) films cured to glass phase had distinctive fracture mechanics; that is, the conjoined films had greater cohesive strength than comparable double thickness or stacked films. Although the mechanism is unclear, it is speculated that this fracture resistance or toughness is a cross-grain effect. In this interpretation, the mechanical stress produced a crack that spanned one layer, but the propagation of the crack was constrained by the conjoined layer. The bond between the conjoined films was crucial as two-layers of stacked films demonstrated a different (bimodal) fracture pattern and significantly lower work of cohesion. Similarly, a double thickness film, likely demonstrating uniform polymer orientation in the glass phase, also demonstrated lower work of cohesion. These results suggest that a random orientation of conjoined films—bonded at the polymer-polymer interface—produced this cross-grain effect and the emergent physical properties of the blended films.

### Example 4—Synthesis of Pectin Film Sealants

### Pectin

The unstandardized citrus pectins used in this study were obtained from a commercial source (Cargill, Minneapolis, MN, USA). Characterization of the high methoxyl citrus pectin was performed. Briefly, the proportion of galacturonic acid residues in the methyl ester form determined the degree of methoxylation. The high-methoxyl pectins (HMP) used in this study demonstrated a greater than 50% degree of methoxylation. The pectin powder was stored in low humidity at 25° C.

### Control Biopolymers

Nanocellulose fibers (NCF) powder, obtained from the University of Maine (Process Development Center, Orono, ME, USA) was dissolved at 25° C. by a controlled increase in water. Both NCF and carboxymethylcellulose (CMC) dissolution was obtained with progressive hydration followed by high-shear mixing (e.g., about 10,000 revolutions per minute (RPM)) using a rotor-stator mixer (L5M-A, Silverson). The dissolved NCF and CMC was poured into standardized molds and cured for further studies. The sodium hyaluronate/CMC polymer was obtained from commercial sources (SepraFilm, Genzyme, Cambridge, MA, USA).

### Spectrophotometry Measurements

Color measurements of the pectin tape (i.e., the pectin film sealant) was obtained with a Minolta spectrophotometer (CM-508d, Minolta, Ramsey, NJ) with a pulsed xenon arc light source. The spectrophotometer had a 400-nanometer (nm) to 700-nm wavelength range at a 20-nm pitch. Light transmittance was calibrated with a white standard; multi-color cellophane reference standards (Outus, Canada) were used for comparison.

### Amsler Grid

Visual distortion from the pectin tape was assessed by projecting a pectin film over half of a black and white Amsler grid. Images were obtained with a 12 megapixel (MP), color camera at a variety of focal lengths without magnification.

### Adhesion Testing

Pectin-cornea adhesion experiments were performed with a custom fixture designed for the TA.XTplus with 5 kg load cell (Stable Micro Systems, Godalming, Surrey, UK). The fixture was composed of a 30 mm diameter flat-ended stainless steel cylindrical probe with pectin mounted to the surface with double-sided proprietary adhesive (Research Division, 3M, St. Paul, MN). The cornea was mounted to a hemispherical foam mount (21 mm diameter) with cyanoacrylate (Vetbond™, 3M™, MN, USA). The cylindrical probe descended onto the cornea at a velocity of 0.5 mm/sec to a compression force of 5 N. The compression force was maintained for a variable development time followed by probe withdrawal at 0.5 mm/sec. Force and distance recordings were obtained at 500 pulses per second (pps).

### Peel Force Testing

Pectin-cornea peel adhesion experiments were performed with a custom fixture designed for the TA.XTplus with 5 kg load cell (Stable Micro Systems). The cornea was mounted to a hemispherical foam mount (21 mm diameter) with cyanoacrylate (Vetbond™). The pectin-corneal adhesion was established with a minimum 60 second development time. The adherent pectin tape was withdrawn at 0.5 mm/sec with real-time video recording. The simultaneous withdrawal force and peel angle recordings were obtained at 500 pps.

### Transcorneal Pressure Testing

Similar to commercial pressure decay leak testing, the anterior chamber of the eye was cannulated with a 14 g catheter (Angiocath, BD Insyte, Sandy, Utah) and sealed with cyanoacrylate (Vetbond™). A clear corneal incision was created remote to the catheter. The anterior chamber was exposed to stepped plateau pressures at 5 mmHg increments. The plateau pressure was monitored for 20 seconds. A stable plateau pressure within 1 mmHg was required to for an additional 5 mmHg increase. After establishing the baseline control, the pectin tape was applied, and the sequence was repeated until a loss of the pressure plateau was identified.

### Scanning Electron Microscopy

After coating the pectin films with 20-25 angstroms (Å) layer of gold in an argon atmosphere, the pectin films were imaged using a Philips XL30 ESEM scanning electron microscope (Philips, Eindhoven, Netherlands) at resolution of 15 kiloelectronvolt (keV) and 21 microangstroms (μÅ). Distance calibration was integrated into standardized automation.

### Statistical Analysis

The statistical analysis was based on measurements in at least three different samples. The unpaired Student's t-test for samples of unequal variances was used to calculate statistical significance. The data was expressed as mean plus one standard deviation (SD). The significance level for the sample distribution was defined as p<0.05.

### Example 5—Physical Properties of Pectin Film Sealant

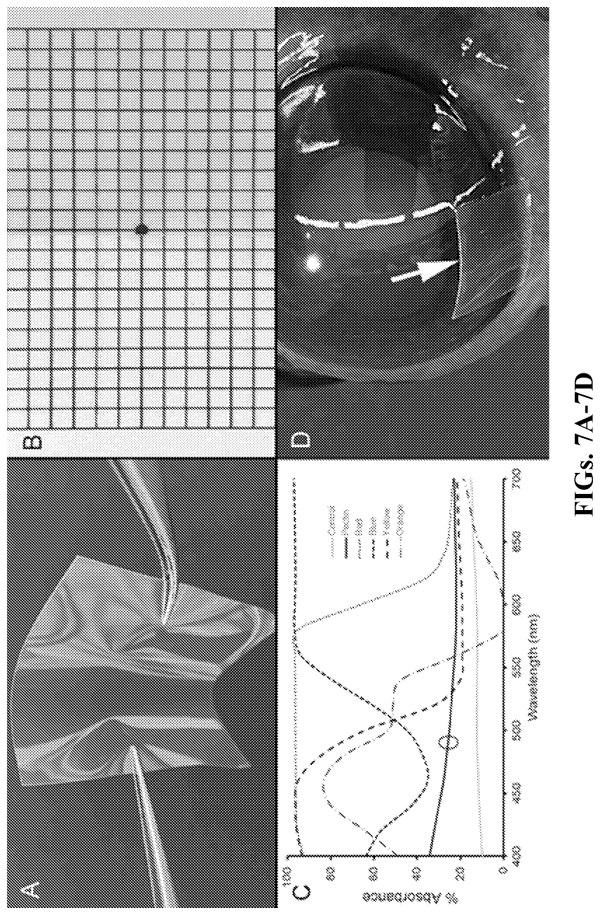

In these studies, the pectin tape was cured as a translucent and flexible 80 (micron) mm thick rectangular strip (FIG. 7A). Potentially relevant for corneal application, the pectin tape demonstrated slight yellow coloration and minimal optical distortion (FIGS. 7B, 7C). On the surface of the cornea, the pectin tape was strongly adherent to the corneal surface within seconds of application (FIG. 7D).

### Pectin-Corneal Tensile Adhesion Strength

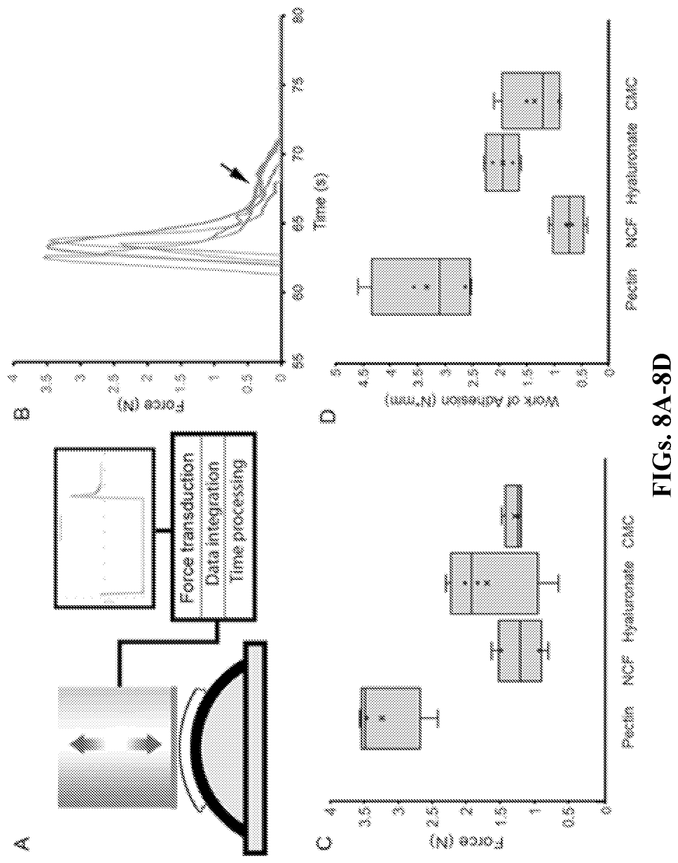

To investigate pectin adhesion to the bovine cornea, pectin tape and the bovine cornea were mounted on custom fixtures designed for an TA-XT Plus material analyzer (Stable Micro Systems) (FIG. 8A). The pectin tape engaged the cornea with a compression force of 5 N for development time of 60 seconds followed by probe withdrawal (FIG. 8A). The force required for probe withdrawal was recorded at 500 pps. The pectin-corneal adhesion was uniformly greater than 3 N with evidence of a distinct debonding curve (FIG. 8B, arrow). In contrast, control biopolymers including NCF, sodium hyaluronate, and CMC demonstrated significantly lower adhesion strength (FIG. 8C, p<0.05) and work of adhesion (FIG. 2D, p<0.05). Systematic variation of development time demonstrated greater than 80% maximal adhesion was obtained within 5 seconds of contact.

### Peel Strength of Pectin-Corneal Adhesion

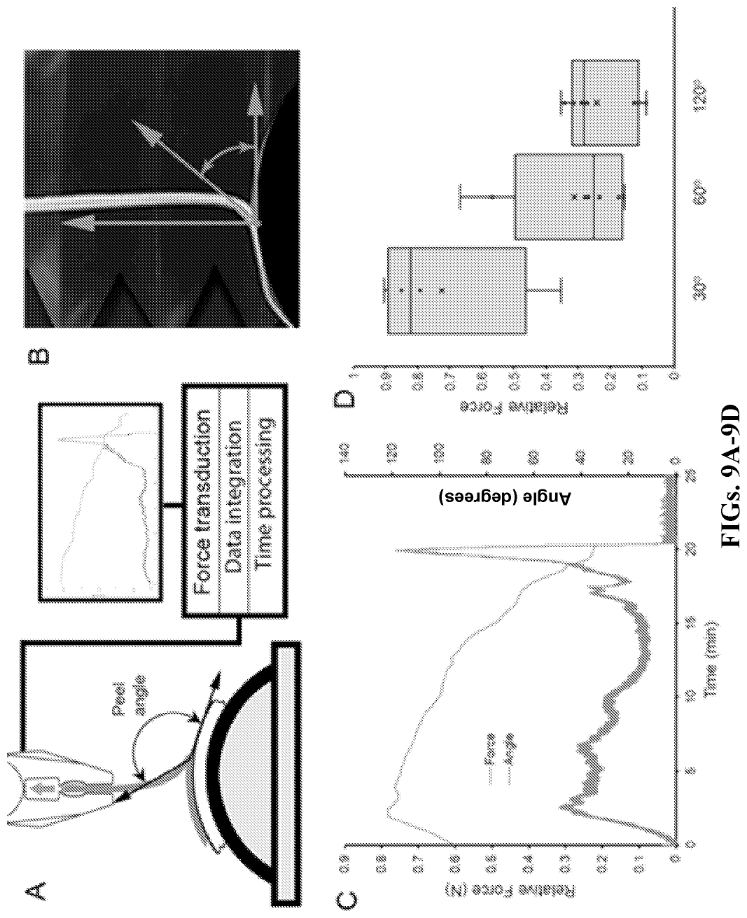

Wound tapes require low-angle peel strength to facilitate reinforcement across linear incisions. To assess peel adhesive strength at a variety of peel angles, real-time force and interface angles at peel rates of 0.5 mm/sec were measured (FIGS. 9A-9C) Maximal peel resistance of the pectin tapes was demonstrated at low peel angles (FIG. 9D).

### Pressure Testing of Pectin-Corneal Adhesion

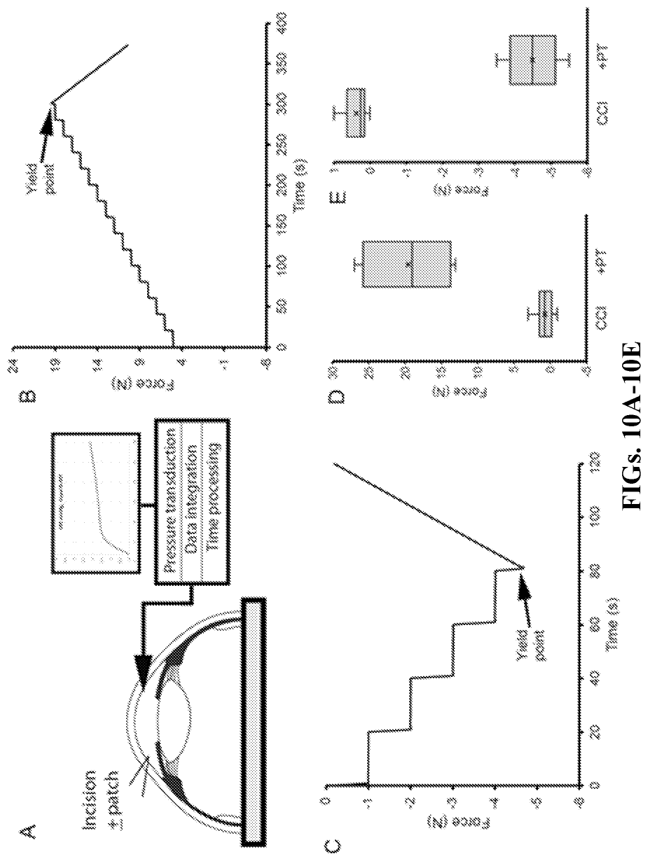

To test outflow resistance of the pectin tape after a corneal incision, the anterior chamber of the bovine globes was cannulated and progressively pressurized until a wound leak was detected (FIG. 10A). The pressurization pattern was an incremental 5 mmHg step with an intervening 20 second (s) plateau; the system was sensitive to ±2 mmHg. The simulated clear corneal incision (CCI) in the bovine cornea without pectin tape failed to generate a pressure greater than 50 mmHg (FIG. 10B). In contrast, the incision sealed with the pectin tape resulted in a pressure range consistently greater than 200 mmHg (214+68.6 mmHg) (FIG. 10B). To test inflow resistance of the pectin tape, the anterior chamber of the bovine cornea was mounted in a custom pressure chamber. The depressurization pattern was an incremental 5 mmHg incremental decrease in pressure with an intervening 20 s plateau. In this simulacrum, the pectin tape reliably sealed transcortical pressure gradients greater than 51.3+8.9 mmHg (FIG. 10).

### Scanning Electron Microscopy (SEM) of Pectin-Corneal Adhesion

To demonstrate the physical interaction of pectin tape with the corneal surface, pectin tape was applied to the bovine cornea and imaged with SEM. SEM demonstrated intimate adhesion of the pectin to the corneal epithelium (FIGS. 5A-5D). The SEM images demonstrated intimate adhesion without detectable interfacial gaps or separation (FIG. 5D, arrows).

### Example 6—Pectin Bioadhesion to the Cornea

In this report, the physical and mechanical properties of pectin adhesion to the bovine cornea were demonstrated. The low-profile pectin tape was translucent, flexible, fracture-resistant, and bioabsorbable. Here, it was shown that pectin tape was also rapidly and strongly adherent to the corneal surface—effectively sealing corneal incisions. Thus, pectin tape may be a potentially useful adjunct in corneal surgery.

Pectin is a structural heteropolysaccharide that comprises approximately 30% of the primary cell walls of plants. One of the most complex polymers on earth, pectin has unique chemical and structural features. Chemically, pectin consists mainly of esterified D-galacturonic acid residues in (1→4) chains. Pectin is bioabsorbable and widely recognized as a harmless food additive in North America and Europe. In the United States, pectin is affirmed GRAS (Generally Recognized as Safe) as defined in the Code of Federal Regulations (21CFR184.1588).

Structurally, pectin is composed of branched polysaccharide chains. In the middle lamella between plant cells, these branched chains entangle with other pectin chains and cellulose microfibrils. The physical entanglement between polymer chains appears to explain the bioadhesive function of pectin. In mammals, pectin has been shown to entangle with the surface glycocalyx of visceral organs including the lung, heart, liver, and bowel. Although there is limited structural data on the corneal epithelial glycocalyx, lectin staining and limited morphologic studies suggest that the corneal glycocalyx is similar to visceral organs. The data presented here, demonstrating strong pectin bioadhesion to the cornea, is consistent with the cornea and visceral organs sharing a similar glycocalyceal structure.

### Other Embodiments

It is to be understood that while certain embodiments have been described within the detailed description, the present disclosure is intended to illustrate and not limit the scope of any embodiment defined by the scope of the appended claims. Other aspects, advantages, and modifications are within the scope of the appended claims.

1.-31. (canceled)

32. A bioadhesive polymer composition comprising:
a first film comprising a polymer comprising: i) high-methoxyl pectin (HMP) and ii) an initial water content ranging from about 37% to about 43% (w/w); and
a second film comprising a polymer comprising: i) HMP and ii) an initial water content ranging from about 9% to about 13% (w/w),
wherein the first and second films are adhered to each other, and
wherein the first film is in a gel phase and the second film is in a glass phase.

33. The bioadhesive polymer composition of claim 32, wherein the HMP is present at a concentration of about 50% (w/w) to about 100% (w/w) in each of the first and second films.

34. The bioadhesive polymer composition of claim 33, wherein an adhesivity of the first and second films to each other ranges from about 22 newton (N) to about 28 N.

35. The bioadhesive polymer composition of claim 32, wherein the first film and the second film have a cross-grain texture at an interface between the first and second films.

36. The bioadhesive polymer composition of claim 32, wherein the initial water content of the first and second films is a water content before and during an initial period of time after the first and second films are adhered to each other, and wherein the initial period of time is about 10 minutes at most.

37. The bioadhesive polymer composition of claim 36, wherein a final water content of the first and second films is a water content after the initial period of time, and wherein the final water content ranges from about 20% to about 30% (w/w).

38. The bioadhesive polymer composition of claim 32, wherein the first and second films exhibit interdiffusion of water, and wherein the first and second films exhibit interdiffusion of HMP.

39. The bioadhesive polymer composition of claim 32, wherein a diffusion coefficient of water through the first and second films is about 2.5-fold to about 5-fold slower than a self-diffusion coefficient of water.

40. The bioadhesive polymer composition of claim 32, wherein a water content at an interface between the first and second films differs from the water content elsewhere in a bulk of the first and second films.

41. The bioadhesive polymer composition of claim 32, wherein the first and second films have a work of cohesion ranging from about 100 J/m² to about 150 J/m², and wherein the first film has thickness of about 62 μm, and the second film has thickness of about 40 μm.

42. A method of preparing a conjoined bioadhesive polymer composition, the method comprising:
providing a first film comprising a polymer comprising: i) high-methoxyl pectin (HMP) and ii) an initial water content ranging from about 37% to about 43% (w/w);
providing a second film comprising a polymer comprising: i) HMP and ii) an initial water content ranging from about 9% to about 13% (w/w); and
compressing the first film and the second film, thereby producing the conjoined bioadhesive polymer composition,
wherein the first film is in a gel phase and the second film is in a glass phase.

43. The method of claim 42, wherein the HMP is present at a concentration of about 50% (w/w) to about 100% (w/w) in each of the first and second films, and wherein each of the first and second films has a water content ranging from about 20% to about 30% (w/w) after the compressing step.

44. The method of claim 42, wherein the first and second films are compressed with a force of about 10 N to about 80 N, and wherein the first and second films are compressed for about 10 seconds to about 12 hours.

45. A method of sealing an ocular injury in an eye of a subject, the method comprising:
providing a bioadhesive film comprising a polymer comprising: i) high-methoxyl pectin (HMP) and ii) water; and
contacting the eye of the subject with the bioadhesive film; and
applying pressure to the bioadhesive film, thereby sealing the ocular injury in the eye of the subject.

46. The method of claim 45, wherein the HMP is present at a concentration of about 50% (w/w) to about 100% (w/w), and wherein the water is present at a concentration of about 10% (w/w) to about 30% (w/w).

47. The method of claim 46, wherein the bioadhesive film reaches 80% of maximal adhesion to the eye within at least about 5 seconds of contact, and wherein the ocular injury is a corneal incision or an injury caused by an ocular surgery.

48. The method of claim 45, wherein the bioadhesive film is flexible, translucent, bioabsorbable, fracture-resistant, or any combination thereof, and wherein the bioadhesive film exhibits minimal optical distortion.

49. The method of claim 45, wherein the bioadhesive film adheres to the eye of the subject with an adhesion strength of at least about 3 N, and wherein the bioadhesive film has a thickness of about 40 μm to about 200 μm.

50. The method of claim 45, wherein, after the bioadhesive film seals the ocular injury, the eye is resistant to a pressure fluctuation ranging from about 50 mmHg to about 280 mmHg.

51. The method of claim 45, wherein the bioadhesive film further comprises a therapeutic agent, and wherein the therapeutic agent is an anti-bacterial agent, an anti-inflammatory agent, a growth factor, or any combination thereof.
